(19) United States
(12) Patent Application Publication
(10) Pub. No.: US 2024/0409666 A1
(43) Pub. Date: Dec. 12, 2024

(54) Polypeptides That Bind to von Willebrand Factor (VWF) A1 Domain or an Autoinhibitory Module, Variants, and Uses Thereof

(71) Applicant: Renhao Li, Atlanta, GA (US)

(72) Inventor: Renhao Li, Atlanta, GA (US)

(21) Appl. No.: 18/717,977

(22) PCT Filed: Dec. 9, 2022
(86) PCT No.: PCT/US2022/081225

§ 371 Date: Jun. 7, 2024

Related U.S. Application Data
(60) Provisional application No. 63/288,115, filed on Dec. 10, 2021.

(51) Int. Cl. C07K 16/36; C07K 9/00

(52) U.S. Cl. CPC C07K 16/36; C07K 9/00; C07K 2317/22; C07K 2317/569; C07K 2317/75; C07K 2317/76

(57) ABSTRACT

This disclosure relates to polypeptides comprising an immunoglobulin single variable domain that specifically binds an autoinhibitory module on the C terminal end or on the N terminal end of von Willebrand Factor (VWF) A1 domain or the von Willebrand Factor (VWF) A1 domain. In certain embodiments this disclosure relates to uses of polypeptides disclosed herein for treating or preventing bleeding or abnormal blood clotting and diseases or conditions related thereto.

### CROSS-REFERENCE TO RELATED APPLICATIONS

This application claims the benefit of U.S. Provisional Application No. 63/288,115 filed Dec. 10, 2021. The entirety of this application is hereby incorporated by reference for all purposes.

### STATEMENT REGARDING FEDERALLY SPONSORED RESEARCH OR DEVELOPMENT

This invention was made with government support under HL143794 and HL154656 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21171PCT.xml. The XML file is 58 KB, was created on Dec. 7, 2022, and is being submitted electronically via the USPTO patent electronic filing system.

### BACKGROUND

The plasma blood clotting protein von Willebrand Factor (VWF) mediates the recruitment of circulating platelets to damaged vessel walls by binding to platelet glycoprotein GPIbα. In this process, GPIb-α binds to the domain A1 contained within VWF. Exposure of the A1 domain occurs under conditions of shear stress resulting in platelet adhesion and eventually blood clots as needed to prevent excessive bleeding.

Thrombotic thrombocytopenia purpura (“TTP”) is a life-threatening disease of the blood coagulation system resulting in undesirable blood clotting. Abnormal platelet aggregation typically damages the kidneys and other organs. Many patients with TTP have a low number of circulating platelets and autoantibodies against ADAMTS13, a protease that cleaves the A2 domain of VWF. ADAMTS13 activity is often deficient in hereditary TTP as well as acquired idiopathic TTP. Plasma transfusions are one method used to prevent blood clotting in patients with TTP. However, this treatment is not ideal as it requires multiple exchanges and transfusions over many days. Treatment is not universally effective. Thus, there is a need to identify improved therapies.

Caplacizumab is a dimerized immunoglobulin single variable domain (ISVD) antibody reported to specifically bind von Willebrand factor. Sully et al. report among patients with TTP, treatment with caplacizumab was associated with a normalization of platelet counts, a reduced incidence of TTP-related death, recurrence of TTP, and thromboembolic events. N Engl J Med, 2019, 380:335-346.

Immunoglobulin single variable domains (ISVDs) against VWF are reported in U.S. Pat. Nos. 10,919,980 and 11,1425,69, WO2004/015425, WO2004/062551, WO2006/074947, WO2006/122825, WO2009/115614 and WO2011/067160.

De Luca et al. report structure and function of the von Willebrand factor A1 domain and analysis with monoclonal antibodies reveals distinct binding sites involved in recognition of the platelet membrane glycoprotein Ib-IX-V complex and ristocetin-dependent activation. Blood, 2000, 95 (1): 164-72.

Arce et al. report activation of von Willebrand factor via mechanical unfolding of its discontinuous autoinhibitory module (AIM) flanking A1. Nat Commun, 2021, 12:236.

References disclosed herein are not an admission of prior art.

### SUMMARY

This disclosure relates to polypeptides comprising an immunoglobulin single variable domain that specifically binds A1 or an autoinhibitory module on the C terminal end or on the N terminal end of von Willebrand Factor (VWF) A1 domain. In certain embodiments this disclosure relates to uses of polypeptides disclosed herein for treating or preventing bleeding, abnormal blood clotting, excessive bleeding, and diseases or conditions related thereto.

In certain embodiments, the immunoglobulin single variable domain (ISVD) has complementary determining region (CDR) 1, CDR 2, and CDR 3 and four framework regions (FRs) derived from camelid antibodies as disclosed herein, and variants thereof. In certain embodiments, the variants are humanizing mutations.

In certain embodiments, the immunoglobulin single variable domain specifically binds the A1 domain or the autoinhibitory module on the C terminal end of A1 and is derived from amino acid sequences in llama antibodies R12NdB2, R6Nd4, or R6Nd6.

In certain embodiments, the polypeptide has the CDR1 of camelid antibody R12NdB2 with the amino acid sequence of EKLTQYVV (SEQ ID NO: 8), CDR2 of SISRSGVFTN (SEQ ID NO: 9); and CDR3 of DSRYSGTDWRVRGE (SEQ ID NO: 10). In certain embodiments, the polypeptide has greater than 85% identity to

(SEQ ID NO: 2)
QVQLVESGGGLVQAGGSLRLSCAASGEKLTQYVVGWLRQAPGKEREFVA
SISRSGVFTNYADSVKGRFTTSKDNAKNTVYLQMNSLTPDDTAIYFCAA
DSRYSGTDWRVRGEYWGQGTQVTVSS.

In certain embodiments, the polypeptide has the CDR1 of camelid antibody R6Nd4 with the amino acid sequence of SRFSSRPMA (SEQ ID NO: 11) and CDR2 of YINWSGGSKY (SEQ ID NO: 12); and a CDR3 of GRAYSAVAVTPRGYD (SEQ ID NO: 13). In certain embodiments, the polypeptide has greater than 85% identity to

(SEQ ID NO: 3)
QVQLVESGGGLVQAGGSLRLSCAASGSRFSSRPMAWFRQTPGKEHDFVA
YINWSGGSKYYADSVKGRFTISRDNAKNTVYLQMDSLKPEDTSIYYCAA
GRAYSAVAVTPRGYDFWGQGTQVTVSS.

In certain embodiments, the polypeptide has the CDR1 of camelid antibody R6Nd6 with the amino acid sequence of IIFSVYHMG (SEQ ID NO: 14), CDR2 of LISLSGSSTD (SEQ ID NO: 15); and CDR3 of RLGSSWK (SEQ ID NO: 16). In certain embodiments, the polypeptide has greater than 85% identity to

(SEQ ID NO: 4)
QVQLVESGGGLVQAGGSLRLSCATSGIIFSVYHMGWFRQTPGKERELVA
LISLSGSSTDYADSVKGRFAISRDNAKDTVFLQMNTLKPEDTAVYYCAA
RLGSSWKYWGQGTQVTVSS.

In certain embodiments, variants are such that the FRs have greater than 70%, 80%, or 85% identity to one or more or all of the FRs in the parent sequence and/or the CDRs have greater than 70%, 80%, or 85% identity to one or more or all of the CDRs in the parent sequence.

In certain embodiments, the polypeptides disclosed herein comprising two immunoglobulin single variable domains conjugated by a peptide linker or a disulfide bond from two cysteine amino acids within each of the two polypeptides.

In certain embodiments, this disclosure relates to methods of treating or preventing blood clotting comprising administering an effective amount of a polypeptide disclosed herein to a subject in need thereof. In certain embodiments, the subject is diagnosed with a disease or condition associated with abnormal blood clotting. In certain embodiments, disease or condition is thrombotic thrombocytopeniarpura (TTP). In certain embodiments, subject is diagnosed with hereditary TTP, acquired idiopathic TTP, or autoantibodies to ADAMTS13.

In certain embodiments, this disclosure relates to methods of providing a reduced incidence of TTP-related death, recurrence of TTP, and other thromboembolic events comprising administering an effective amount of a polypeptide disclosed herein to a subject in need thereof. In certain embodiments, this disclosure relates to methods of normalizing platelet counts comprising administering an effective amount of a polypeptide disclosed herein to a subject in need thereof.

In certain embodiments, the polypeptide comprises an immunoglobulin single variable domain that specifically binds the A1 domain or autoinhibitory module on the N terminal end of A1 and is derived from amino acid sequences in llama antibodies R12CdB9 or variants thereof. In certain embodiments, the CDR1 has the amino acid sequence of LTFMDHVM (SEQ ID NO: 5) and a CDR2 that is AVGRSAIMRD (SEQ ID NO: 6); and a CDR3 that is RTPFPSDMTWSLPNDYI (SEQ ID NO:7). In certain embodiments, the polypeptide has greater than 85% identity to

(SEQ ID NO: 1)
QVQLVESGGGLVQAGGSLRLSCAASGLTFMDHVMGWFRQAPGKEREFVA
AVGRSAIMRDYADLVKGRFTISRDNAKNTVYLQMDSLKFEDTAVYYCAA
RTPFPSDMTWSLPNDYIYWGQGAQVTVSS.

In certain embodiments, variants are such that FRs have greater than 70%, 80%, or 85% identity to the FRs in the parent sequence and or the CDRs have greater than 70%, 80%, or 85% identity to the CDRs in the parent sequence.

In certain embodiments, the polypeptide comprises an immunoglobulin single variable domain that specifically binds A1 or the autoinhibitory module on the N terminal end of the A1 domain in von Willebrand Factor (VWF) having complementary determining region (CDR) 1, CDR 2, and CDR 3 and framework regions (FRs), and wherein the CDR1, CDR 2, and CDR 3 are from camelid antibodies Cd1C4, Cd1C11, Cd1D12 or variants thereof.

In certain embodiments, the polypeptide has the CDR1 of camelid antibody Cd1C4 with the amino acid sequence of RAFSQYSV (SEQ ID NO: 17), CDR2 of NWSGTKA (SEQ ID NO: 18); and CDR3 of HRGLYYEGTNYSQKD (SEQ ID NO: 19). In certain embodiments, the polypeptide has greater than 85% identity to

(SEQ ID NO: 20)
QVQLVESGGGLVQTGGSLTLSCAASGRAFSQYSVGWFRQAPGKERTFVA
AINWSGTKAYYAGSVKGRSTISRDRAKTTVFLQMNSLKPEDTAVYYCAT
HRGLYYEGTNYSQKDEYDYWGQGTQVTVSS.

In certain embodiments, the polypeptide has the CDR1 of camelid antibody Cd1C11 with the amino acid sequence of RTVSHYSV (SEQ ID NO: 21), CDR2 of NWSGDKA (SEQ ID NO: 22); and CDR3 of RRGLYYEGTDYSRKD (SEQ ID NO: 23). In certain embodiments, the polypeptide has greater than 85% identity to

(SEQ ID NO: 24)
QVQLVESGGGLVQTGGSLRLSCAASGRTVSHYSVGWFRQAPGKERVFVG
AINWSGDKAYYVGSVKGRSTIRRDNAKNTVYLQMNSLKPEDTAVYYCAT
RRGLYYEGTDYSRKDEYDYWGQGTQVTVSS.

In certain embodiments, the polypeptide has the CDR1 of camelid antibody Cd1C12 having CDR1 with the amino acid sequence of FTLDDYAI (SEQ ID NO: 25), CDR2 of NWSGTKA (SEQ ID NO: 26); and CDR3 of RGLHYGGINYSQKD (SEQ ID NO: 27). In certain embodiments, the polypeptide has greater than 85% identity to

(SEQ ID NO: 28)
QVQLVESGGGLVQPGGSLRLSCAASGFTLDDYAIGWFRQVPGKERTFVG
AINWSGTKAYYAGSVKGRSTVRRDNAKNTVYLQMNSLKPEDTAVYYCAT
HRGLHYGGINYSQKDEYDYWGQGTQVTVSS.

In certain embodiments, variants are such that the FRs have greater than 70%, 80%, or 85% identity to one or more or all of the FRs in the parent sequence and or the CDRs have greater than 70%, 80%, or 85% identity to one or more or all of the CDRs in the parent sequence.

In certain embodiments, this disclosure relates to polypeptides or other agents that specifically bind SPTTLYVEDISEP (SEQ ID NO: 64), fragment, or O-glycosylated (sialic acid O-sialylated) substituted derivative for uses disclosed herein. In certain embodiments, the polypeptide is a nanobody or antibody.

In certain embodiments, this disclosure relates to methods of treating or preventing spontaneous bleeding comprising administering an effective amount of a platelet clotting activating polypeptide disclosed herein to a subject in need thereof.

In certain embodiments, the subject is exhibiting symptoms of, at risk of, or diagnosed with von Willebrand Disease (VWD), thrombocytopenia, or hemophilia. In certain embodiments, the subject is at risk of, exhibiting symptoms of, or diagnosed with thrombocytopenia caused by bone marrow dysfunctions or chemotherapy treatments. In certain embodiments, the platelet clotting activating polypeptide disclosed herein is administered prior to, after, or during a surgical procedure, e.g., organ, tissue or cell transplantation, stem cell transplantation, extracorporeal membrane oxygenation.

In certain embodiments, this disclosure relates to methods of using polypeptides disclosed herein that bind VWF in assays. In certain embodiments, this disclosure relates to methods of measuring the ability of VWF to aggregate platelets in the presence of a platelet clotting activating polypeptide disclosed herein, comprising contacting the polypeptide, a sample comprising VWF and platelets, and thereafter measuring platelet aggregation.

In certain embodiments, this disclosure relates to pharmaceutical compositions comprising a polypeptide or vector encoding the same disclosed herein and a pharmaceutically acceptable excipient for uses reported herein.

In certain embodiments, this disclosure relates to nucleic acids or vectors encoding a polypeptide disclosed herein. In certain embodiments, this disclosure relates to expression system or somatic cell comprising a nucleic acid or vector encoding a polypeptide disclosed herein.

In certain embodiments, this disclosure relates to polypeptides disclosed herein, or nucleic acids or vectors encoding the same, for use in production of a medicament to treat or prevent blood clotting, abnormal blood clotting, excessive bleeding, or thrombosis, cardiovascular disease, and other diseases or conditions reported herein. In certain embodiments, the nucleic acid is DNA or RNA.

### BRIEF DESCRIPTION OF THE SEVERAL VIEWS OF THE DRAWINGS

FIG. 1A shows a schematic of a VWF monomer with binding sites of natural proteins such as GPIb-α and ADAMTS13. Also illustrated are the N and C terminal discontinuous autoinhibitory module (AIM) flanking A1. Alignments with various AIM-A1 fragments are shown.

FIG. 1B shows the sequence (SEQ ID NO: 29) of a recombinant protein AIM-A1 prepared for experiments reported herein. NAIM is QEPGGLVVPPTDAPVSPTTLYVEDISE PPLHDFY (SEQ ID NO: 30) and CAIM is DLAPEAPPPTLPPDMAQVTVGPGLLGVST LGPKRN (SEQ ID NO: 31).

FIG. 2 illustrates a molecular model of force induced VWF A1 domain activation via dissolution of the AIM. During hemostasis, only above a critical force (Fcrit), will the AIM unfold to expose the A1 domain for GPIbα binding. VWF bearing type 2B mutations, binding to ristocetin, or mAb 6G1 lowers the critical unfolding force of the AIM, allowing GPIbα to bind under lower tensile forces. VWF bound to VHH81 (mono ISVD of caplacizumab) is able to withstand forces that would normally activate A1 and increases the critical unfolding force of the AIM.

FIG. 3 shows biolayer interferometry traces indicating that Nd4 (left panel) and Nd6 (right) ISVD antibodies bind to the AIM-A1 protein.

FIG. 4 shows platelet-rich plasma (PRP) aggregometry traces showing the inhibition of ristocetin-induced platelet aggregation the ISVDs. 1 μM ISVD was added to the mix, and 1.5 mg/ml ristocetin was used.

FIG. 5A shows a sequence comparison of R6Nd4 (Top) (SEQ ID NO: 3) and VHH81 (Bottom) (SEQ ID NO: 32).

FIG. 5B shows a sequence comparison of R6Nd6 (Top) (SEQ ID NO: 4) and VHH81 (Bottom) (SEQ ID NO: 32).

FIG. 6A shows biolayer interferometry traces that demonstrate binding of ISVD antibodies to the AIM-A1 protein.

FIG. 6B shows platelet-rich plasma aggregometry traces indicating the ISVD antibodies agglutinate platelets.

FIG. 7A shows data indicating 6D12 binds to glycosylated AIM-A1.

FIG. 7B shows data indicating 6D12 exhibited binding to NAIM-Fc with a binding affinity of 3.2 nM.

FIG. 7C shows data indicating 6D12 exhibited binding to 1253-1266-Fc with a binding affinity of 4.3 nM.

FIG. 7D shows data indicating 6D12 exhibited binding to desialylated NAIM-Fc, produced from neuraminidase treatment of NAIM-Fc, with an affinity of 32 nM, which is weaker than that for NAIM-Fc.

### DETAILED DISCUSSION

Before the present disclosure is described in greater detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be limited only by the appended claims. Although the function of certain compositions disclosed herein are believed to operate by particular mechanisms, it is not intended that embodiments of this disclosure be limited by any specific mechanism.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

### Polypeptides

The terms “protein,” “peptide,” and “polypeptide” refer to polymers comprising amino acids joined via peptide bonds and are used interchangeably. Amino acids may be naturally or non-naturally occurring. A “chimeric protein” or “fusion protein” is a molecule in which different portions of the protein are derived from different origins such that the entire molecule is not naturally occurring. A chimeric protein may contain amino acid sequences from the same species or different species as long as they are not arranged together in the same way that they exist in a natural state. Examples of a chimeric protein include sequences disclosed herein that contain one, two or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, a polyhistidine (HIS) tag, e.g., typically four or more histidine amino acids, a human influenza hemagglutinin (HA) tag, a TAT polypeptide, GST peptide, or a self-cleaving peptide P2A-GSG.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide consists of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide consists of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

A “variant” refers to a chemically similar sequence because of amino acid changes. In certain embodiments, a variant contains one or two, or more amino acid deletions or substitutions. In certain embodiments, the substitutions are conserved substitutions. In certain embodiments, a variant contains one, two, or ten or more, or ten or less amino acid additions. In certain embodiments, the additions may be to the N-terminus or the C-terminus. The variant may be substituted with one or more chemical substituents.

A conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. A variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted, or deleted without abolishing biological activity may be found using computer programs well known in the art. Variants can be tested in functional assays. Certain variants have less than 10%, and preferably less than 5%, and still more preferably less than 2% changes (whether substitutions, deletions, and so on). Variants can be prepared for testing by mutating a vector to produce appropriate codon alternatives for peptide translation.

In certain embodiments, the peptides disclosed herein have at least one non-naturally occurring molecular modification, such as the attachment of polyethylene glycol, the attachment of a chimeric peptide, the attachment of a fluorescent dye comprising aromatic groups, fluorescent peptide, a chelating agent capable of binding a radionuclide, e.g., 18F. In certain embodiments, the peptides contain an N-terminal acetyl, propionyl group, myristoyl and palmitoyl, group or N-terminal mono- or di-methylation, or a C-terminal alkyl ester or amide. In certain embodiments, this disclosure contemplates peptides disclosed herein labeled using commercially available biotinylation reagents. Biotinylated peptide can be used in streptavidin/avidin affinity binding, purification, and detection. In certain embodiments, the disclosure contemplates a peptide disclose herein containing azide-derivatives of naturally occurring monosaccharides such as N-azidoacetylglucosamine, N-azidoacetylmannosamine, and N-azidoacetylgalactosamine.

In certain embodiments, this disclosure contemplates derivatives of peptide disclose herein wherein one or more amino acids are substituted with chemical groups to improve pharmacokinetic properties such as solubility and serum half-life, optionally connected through a linker. In certain embodiments, such a derivative may be a prodrug wherein the substituent or linker is biodegradable, or the substituent or linker is not biodegradable. In certain embodiments, contemplated substituents include a saccharide, polysaccharide, acetyl, fatty acid, lipid, and/or polyethylene glycol. The substituent may be covalently bonded through the formation of amide bonds on the C-terminus or N-terminus of the peptide optionally connected through a linker. In certain embodiments, it is contemplated that the substituent may be covalently bonded through an amino acid within the peptide, e.g., through an amine side chain group such as lysine or an amino acid containing a carboxylic acid side chain group such as aspartic acid or glutamic acid, within the peptide comprising a sequence disclosed herein. In certain embodiments, it is contemplated that the substituent may be covalently bonded through a cysteine in a sequence disclosed herein optionally connected through a linker. In certain embodiments, a substituent is connected through a linker that forms a disulfide with a cysteine amino acid side group.

As used herein, a “lipid” group refers to a hydrophobic group that is naturally or non-naturally occurring that is highly insoluble in water. As used herein a lipid group is considered highly insoluble in water when the point of connection on the lipid is replaced with a hydrogen and the resulting compound has a solubility of less than 0.63×10⁻⁴% w/w (at 25° C.) in water, which is the percent solubility of octane in water by weight. See Solvent Recovery Handbook, 2^{nd} Ed, Smallwood, 2002 by Blackwell Science, page 195. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

As used herein, the term “conjugated” refers to linking molecular entities through covalent bonds, linking groups, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge two molecular moieties together. An example formula may be —Rₘ— wherein R is selected individually and independently at each occurrence as: —CRₘRₘ—, —CHRₘ—, —CH—, —C—, —CH₂—, —C(OH)Rₘ, —C(OH)(OH)—, —C(OH)H, —C(Hal)Rₘ—, —C(Hal)(Hal)-, —C(Hal)H—, —C(N₃)Rₘ—, —C(CN)Rₘ—, —C(CN)(CN)—, —C(CN)H—, —C(N₃)(N₃)—, —C(N₃)H—, —O—, —S—, —N—, —NH—, —NRₘ—, (C═O)—, —(C═NH)—, —(C═S)—, —(C═CH₂)—, which may contain single, double, or triple bonds individually and independently between the R groups. If an R is branched with an Rₘ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “m” may be less than 100, or 50, or 25, or 10. Examples of linking groups include bridging alkyl groups and alkoxyalkyl groups. Linking groups may be substituted with one or more substituents.

As used herein, the term “biodegradable” in reference to a substituent or linker refers to a molecular arrangement in a peptide derivative that when administered to a subject, e.g., human, will be broken down by biological mechanism such that a metabolite will be formed and the molecular arrangement will not persist for over a long period of time, e.g., the molecular arrangement will be broken down by the body after a several hours or days. In certain embodiments, the disclosure contemplates that the biodegradable linker or substituent will not exist after a week or a month.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

Polypeptides can be produced by any commonly used method. Typical examples include the recombinant expression in suitable host systems, e.g., bacteria or yeast. In general, the polypeptides may be produced by living host cells that have been genetically engineered to produce the polypeptide. Methods of genetically engineering cells to produce proteins are well known in the art. See e.g. Ausubel et al., eds. (1990), Current Protocols in Molecular Biology (Wiley, New York). Such methods include introducing nucleic acids that encode and allow expression of the polypeptide into host cells. These host cells can be bacterial cells, fungal cells, or animal cells grown in culture. In one embodiment, polypeptides are produced in mammalian cells. Typical mammalian host cells for expressing the clone antibodies or antigen-binding fragments thereof include Chinese Hamster Ovary (CHO cells) (including dhfr-CHO cells, described in Urlaub and Chasin, Proc. Natl. Acad. Sci. USA 77:4216-4220 (1980), used with a DHFR selectable marker, e.g., as described in Kaufman and Sharp, Mol. Biol. 159:601-621 (1982)), lymphocytic cell lines, e.g., NSO myeloma cells and SP2 cells, COS cells, and a cell from a transgenic animal, e.g., a transgenic mammal. For example, the cell is a mammary epithelial cell.

In addition to the nucleic acid sequences encoding the polypeptide, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). For example, typically the selectable marker gene confers resistance to drugs, such as G418, hygromycin, or methotrexate, on a host cell into which the vector has been introduced.

Standard molecular biology techniques can be used to prepare the recombinant expression vector, transfect the host cells, select for transformants, culture the host cells and recover the polypeptides from the culture medium. For example, the polypeptides can be isolated by affinity chromatography.

In certain embodiments, this disclosure relates to nucleotide sequences or nucleic acids that encode the polypeptides disclosed herein, genetic constructs that include the foregoing nucleotide sequences or nucleic acids and one or more elements for genetic constructs known per se. In certain embodiments, this disclosure relates to hosts or host cells that contain such nucleotide sequences or nucleic acids, and/or that express (or are capable of expressing), the polypeptides disclosed herein.

In certain embodiments, this disclosure relates to methods for preparing polypeptides disclosed herein, which method comprises cultivating or maintaining a host cell as described herein under conditions such that said host cell produces or expresses the polypeptides disclosed herein.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression system. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. See, e.g., Shimizu et al., Cell-free translation reconstituted with purified components, 2001, Nat. Biotechnol., 19, 751-755 and Asahara & Chong, Nucleic Acids Research, 2010, 38 (13): e141, both hereby incorporated by reference in their entirety.

A “selectable marker” is a nucleic acid introduced into a recombinant vector that encodes a peptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose: galactose-1-phosphate uridyltransferasel (galT), feedback-insensitive a subunit of anthranilate synthase (OASAID), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable moiety that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous peptide in the receptor. A label includes the incorporation of a radiolabeled amino acid or the covalent attachment of biotinyl moieties to a peptide that can be detected by marked avidin (for example, streptavidin containing a fluorescent marker or enzymatic activity that can be detected by optical or colorimetric methods). Various methods of labeling peptides and glycoproteins are known in the art and may be used. Examples of labels for peptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined peptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms (linking groups) of various lengths to reduce potential steric hindrance.

In certain embodiments, the disclosure relates to recombinant peptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a peptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken β-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar-STNQ.

“Immunoglobulin single variable domains” are antibodies whose complementarity determining regions (CDRs) are part of a single domain polypeptide such as antibodies naturally devoid of light chains, single domain antibodies derived from conventional 4-chain antibodies. For clarity reasons, this variable domain derived from a heavy chain antibody naturally devoid of light chain is known herein as a VHH to distinguish it from the conventional VH of four chain immunoglobulins. Such a VHH molecule can be derived from antibodies raised in Camelidae species, for example in camel, llama, dromedary, alpaca, and guanaco.

With regard to variable chain immunoglobulins, the location of binding complementarity-determining regions (CDRs) sometimes varies depending on the specific sequence context and animal. The CDRs can be determined through epitope studies and sequence alignment comparisons of the constant and framework regions for the specific animal. As is well-known in the art that there are multiple conventions to define and describe the CDRs of a VH or VHH fragment, such as the Kabat definition (which is based on sequence variability) and the Chothia definition (which is based on the location of the structural loop regions).

In general, identifying CDRs can be accomplished utilized the following criteria: for CDR-H1 the start residue is approximately 26 to 30 after the first amino acid and typically 4 after a Cys and typically end with Trp, e.g., Trp-Val, but also, Trp-Ile, Trp-Ala. The length is typically about 6 to 12 residues. CDR-H2 typically starts at about 15 residues after the end of CDR-H1. Residues before the start are typically Trp-Ile-Gly but can be a number of variations, and typical ends with Lys/Arg-Leu/Ile/Val/Phe/Thr/Ala-Thr/Ser/Ile/Ala. The length can vary from about 8 to 20 amino acids; CDR-H3 is typically about 30-33 residues after the end of CDR-H2, and often identified 3 amino acids after a Cys, such as in the example Cys-Ala-Arg. The end is sometimes identified before residues such as Trp-Gly. The length can vary widely, e.g., 4-25 or more depending on the animal.

Immunoglobulin Single Variable Domains that Specifically Bind the A1 Domain or the Autoinhibitory Module on the C Terminal End or on the N Terminal End of the A1 Domain

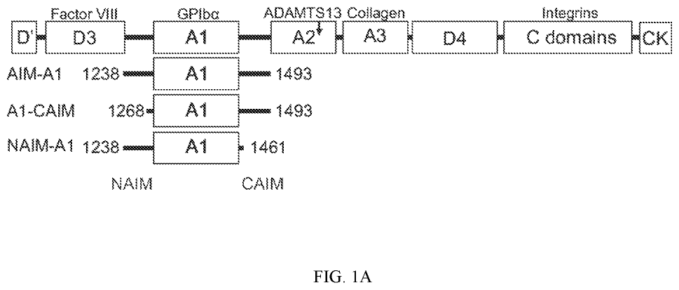

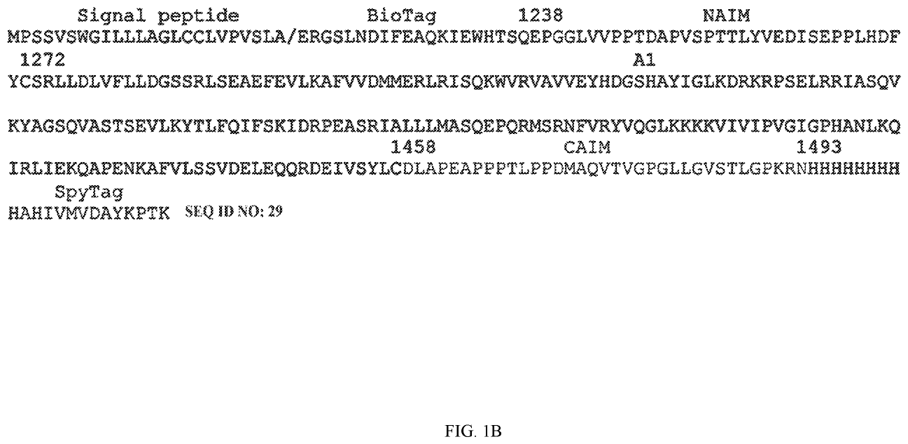

In certain embodiments, this disclosure relates to polypeptides having amino acid sequences that bind to A1 or AIM-A1 protein. In certain embodiments, this disclosure relates to polypeptides that selectively bind the segment of the AIM that is on the N terminal side of the A1 segment (NAIM) or that is on the C terminal side of the A1 segment (CAIM). See FIG. 1A. In certain embodiments, this disclosure relates to immunoglobulin single variable domains (also referred to herein as “ISVDs”), and more in particular heavy-chain immunoglobulin single variable domains, that selectively bind to AIM-A1 protein (NAIM or CAIM) as well as to proteins, polypeptides and other constructs, compounds, molecules, or chemical entities that comprise such. In certain embodiments, the polypeptides of this disclosure are fusion proteins.

In certain embodiments, this disclosure relates to polypeptides containing sequences disclosed herein and variants for the purpose of generating therapeutic products for addressing diseased and conditions reported herein.

In certain embodiments, the polypeptides comprise a sequence of a variable heavy chain identified as R12CdB9 (CDRs bold):

QVQLVESGGGLVQAGGSLRLSCAASGLTFMDHVMGWFRQAPGKEREFVAAVG RSAIMRDYADLVKGRFTISRDNAKNTVYLQMDSLKFEDTAVYYCAARTPFPSDMTWS LPNDYIYWGQGAQVTVSS (SEQ ID NO: 1) or variant thereof. In certain embodiments, the A1 or AIM-A1-binding ISVDs of the disclosure preferably comprise one or more of the following CDRs: a CDR1 that is LTFMDHVM (SEQ ID NO: 5) and a CDR2 that is AVGRSAIMRD (SEQ ID NO: 6); and a CDR3 that is RTPFPSDMTWSLPNDYI (SEQ ID NO:7) or variants thereof.

In certain embodiments, the polypeptides comprise sequence of a variable heavy chain identified as R12NdB2 sequence (CDRs bold):

QVQLVESGGGLVQAGGSLRLSCAASGEKLTQYVVGWLRQAPGKEREFVASISR SGVFTNYADSVKGRFTTSKDNAKNTVYLQMNSLTPDDTAIYFCAADSRYSGTDWRVR GEYWGQGTQVTVSS (SEQ ID NO: 2) or variant thereof. In certain embodiments, the A1 or AIM-A1-binding ISVDs of the disclosure preferably comprise one or more of the following CDRs: a CDR1 that is EKLTQYVV (SEQ ID NO: 8) and a CDR2 that is SISRSGVFTN (SEQ ID NO: 9); and a CDR3 that is DSRYSGTDWRVRGE (SEQ ID NO: 10) or variants thereof.

In certain embodiments, the polypeptides comprise a sequence of a variable heavy chain identified as R6Nd4 sequence (CDRs bold):

QVQLVESGGGLVQAGGSLRLSCAASGSRFSSRPMAWFRQTPGKEHDFVAYINW SGGSKYYADSVKGRFTISRDNAKNTVYLQMDSLKPEDTSIYYCAAGRAYSAVAVTPR GYDFWGQGTQVTVSS (SEQ ID NO: 3) or variant thereof. In certain embodiments, the A1 or AIM-A1-binding ISVDs of the disclosure preferably comprise one or more of the following CDRs: a CDR1 that is SRFSSRPMA (SEQ ID NO: 11) and a CDR2 that is YINWSGGSKY (SEQ ID NO: 12); and a CDR3 that is GRAYSAVAVTPRGYD (SEQ ID NO: 13) or variants thereof.

In certain embodiments, the polypeptides comprise a sequence of a variable heavy chain identified as R6Nd6 sequence (CDRs bold):

QVQLVESGGGLVQAGGSLRLSCATSGIIFSVYHMGWFRQTPGKERELVALISLS GSSTDYADSVKGRFAISRDNAKDTVFLQMNTLKPEDTAVYYCAARLGSSWKYWGQG TQVTVSS (SEQ ID NO: 4) or variant thereof. In certain embodiments, the A1 or AIM-A1-binding ISVDs of the disclosure preferably comprise one or more of the following CDRs: a CDR1 that is IIFSVYHMG (SEQ ID NO: 14) and a CDR2 that is LISLSGSSTD (SEQ ID NO: 15); and a CDR3 that is RLGSSWK (SEQ ID NO: 16) or variants thereof.

In certain embodiments, the polypeptides that specifically bind the A1 domain or autoinhibitory module on the N terminal end of A1 and are derived from amino acid sequences in llama antibodies R12CdB9 or variants thereof.

In certain embodiments, the polypeptides comprise a sequence of a variable heavy chain identified as R12CdB9 sequence (CDRs bold):

QVQLVESGGGLVQAGGSLRLSCAASGLTFMDHVMGWFRQAPGKEREFVAAVG RSAIMRDYADLVKGRFTISRDNAKNTVYLQMDSLKFEDTAVYYCAARTPFPSDMTWS LPNDYIYWGQGAQVTVSS (SEQ ID NO: 1) or variant thereof. In certain embodiments, the A1 or AIM-A1-binding ISVDs of the disclosure preferably comprise one or more of the following CDRs: a CDR1 that is LTFMDHVM (SEQ ID NO: 5) and a CDR2 that is AVGRSAIMRD (SEQ ID NO: 6); and a CDR3 that is RTPFPSDMTWSLPNDYI (SEQ ID NO:7) or variants thereof.

In certain embodiments, the FRs have greater than 85% identity to the FRs in the parent sequence and or the CDRs have greater than 85% identity to the CDRs in the parent sequence.

In certain embodiments, the polypeptides comprise an immunoglobulin single variable domain that specifically binds A1 or the autoinhibitory module on the N terminal end of the A1 domain in von Willebrand Factor (VWF) having complementary determining region (CDR) 1, CDR 2, and CDR 3 and framework regions (FRs), and wherein the CDR1, CDR 2, and CDR 3 are from camelid antibody Cd1C4, Cd1C11, Cd1D12 or variants thereof.

In certain embodiments, the polypeptides comprise a sequence of a variable heavy chain identified as Cd1C4 sequence (CDRs bold):

QVQLVESGGGLVQTGGSLTLSCAASGRAFSQYSVGWFRQAPGKERTFVAAINW SGTKAYYAGSVKGRSTISRDRAKTTVFLQMNSLKPEDTAVYYCATHRGLYYEGTNYS QKDEYDYWGQGTQVTVSS (SEQ ID NO: 20) or variant thereof. In certain embodiments, the A1 or AIM-A1-binding ISVDs of the disclosure preferably comprise one or more of the following CDRs of Cd1C4 having CDR1 with the amino acid sequence of RAFSQYSV (SEQ ID NO: 17), CDR2 of NWSGTKA (SEQ ID NO: 18); and CDR3 of HRGLYYEGTNYSQKD (SEQ ID NO: 19) or variants thereof.

In certain embodiments, the polypeptides comprise a sequence of a variable heavy chain identified as Cd1C11:

QVQLVESGGGLVQTGGSLRLSCAASGRTVSHYSVGWFRQAPGKERVFVGAINW SGDKAYYVGSVKGRSTIRRDNAKNTVYLQMNSLKPEDTAVYYCATRRGLYYEGTDY SRKDEYDYWGQGTQVTVSS (SEQ ID NO: 24) or variant thereof. In certain embodiments, the A1 or AIM-A1-binding ISVDs of the disclosure preferably comprise one or more of the following CDRs or Cd1C11 having CDR1 with the amino acid sequence of RTVSHYSV (SEQ ID NO: 21), CDR2 of NWSGDKA (SEQ ID NO: 22); and CDR3 of RRGLYYEGTDYSRKD (SEQ ID NO: 23) or variants thereof.

In certain embodiments, the polypeptides comprise a sequence of a variable heavy chain identified as Cd1C12:

QVQLVESGGGLVQPGGSLRLSCAASGFTLDDYAIGWFRQVPGKERTFVGAINW SGTKAYYAGSVKGRSTVRRDNAKNTVYLQMNSLKPEDTAVYYCATHRGLHYGGINY SQKDEYDYWGQGTQVTVSS (SEQ ID NO: 28) or variants thereof. In certain embodiments, the A1 or AIM-A1-binding ISVDs of the disclosure preferably comprise one or more of the following CDRs of Cd1C12 having CDR1 with the amino acid sequence of FTLDDYAI (SEQ ID NO: 25), CDR2 of NWSGTKA (SEQ ID NO: 26); and CDR3 of RGLHYGGINYSQKD (SEQ ID NO: 27) or variants thereof.

In certain embodiments, the FRs have greater than 85% identity to one or more or all of the FRs in the parent sequence and or the CDRs have greater than 85% identity to one or more or all of the CDRs in the parent sequence.

In certain embodiments, this disclosure contemplates serum A1 or AIM-A1-binding ISVDs (NAIM or CAIM) of the disclosure preferably bind to (human) A1 or AIM-A1 with an affinity better than 100 nM, preferably better than 50 nM.

In certain embodiments, the A1 or AIM-A1-binding ISVDs of the disclosure and compounds and polypeptides comprising the same (as further described herein) preferably have a half-life (defined as t1/2 beta) in a human that is more than 1 hour, preferably more than 2 hours, more preferably of more than 6 hours, such as of more than 12 hours, and for example of about one day, two days, one week, or two weeks.

In certain embodiments, this disclosure contemplates serum A1 or AIM-A1-binding ISVDs (NAIM or CAIM) that are variants that have reduced binding by interfering factors (generally referred to as “pre-existing antibodies”) that may be present in the sera from some healthy human subjects as well as from diseased patients. Reference is made to WO2012/175741, WO2013/024059, and WO2015/173325).

### Humanized Immunoglobulins

The term, “humanized” refers to a polypeptide containing one or more amino acid mutations so that immunogenicity upon administration in human patients, e.g., due to “pre-existing antibodies”, is reduced, made highly unlikely, or nonexistent. Anaphylaxis is a severe allergic reaction to an allergen, e.g., polypeptide. Non-human proteins contain amino acid residues that may be immunogenic when targeted by preexisting antibodies circulating in a human patient. Thus, it is desirable to mutate residues within a therapeutic protein so that the peptide sequences are similar to peptide sequences that commonly occurs in human proteins, provided that the desirable therapeutic properties are retained, thereby reducing the risk of undesirable allergic reactions. In immunoglobulins, this is typically accomplished by comparing sequences, preferably framework sequences, and identifying amino acid substitutions providing “humanized” sequences frequently found within human immunoglobulins. These humanized polypeptides reduce the risk of undesirable immune reactions providing a polypeptide that is substantially non-immunogenic in humans and retain the affinity and activity of the original polypeptide.

Humanizing amino acid mutations within immunoglobulin single variable domains (ISVDs) are known, See, e.g., U.S. Pat. No. 11,142,569, and this disclosure contemplates that variants of polypeptide sequences disclosed herein contain such mutations. For example, the presence of a C-terminal alanine (or a C-terminal extension generally) can reduce and essentially fully prevent an immune reaction that is the result of the binding of the “pre-existing antibodies” that can be found in the sera from a range of subjects (healthy subjects as patients).

Other examples are provided in FIGS. 5A and 5B which show sequence alignments of VHH81 when compared to R6Nd4 (SEQ ID NO: 3) and R6Nd6 (SEQ ID NO: 4). In certain embodiments, this disclosure contemplates that polypeptides of this disclosure contain CDRs1-3 of and one or more of the same framework mutations contained within VHH81. In certain embodiments, it is contemplated that polypeptides/ISVDs, e.g., R6Nd4 (SEQ ID NO: 3), have one or more or all of the following mutations within one or more of the framework regions: FR1 of Q1E, A13P, FR 2 of T40A, E44G; H45R, D46E, F47L, and FR3, A61P, K65E, N77R, T78M, D84N, K87R, P88A, S92A, 193V, or combinations thereof.

In certain embodiments, it is contemplated that polypeptides/ISVDs, e.g., R6Nd6 (SEQ ID NO: 4), have one or more or all of the following mutations within one or more of the framework regions: FR1 of Q1E, A13P, T24A; FR2 of T40A, E44G; and FR3, A61P, K65E, A69T, D77R, T78M, F80Y, T85S, K87R, P88A, or combinations thereof.

It is also contemplated that humanization of VHH polypeptides includes the introduction and mutagenesis of only a limited number of amino acids in a single polypeptide chain without dramatic loss of binding and/or inhibition activity.

In certain embodiments, polypeptides disclosed herein are contemplated to contain mutations sequences as reported in U.S. Pat. No. 11,142,569 (i.e., mutations compared to the sequences of ISVDs reported herein) such as a 11V, i.e., GGGVV (SEQ ID NO: 33); 93L, i.e., TALYYV (SEQ ID NO: 34), for example, 93T, i.e., TATYY (SEQ ID NO: 35), FR4 K mutations, i.e., VKVSS (SEQ ID NO: 36) or VTVKS (SEQ ID NO: 37), a FR4 Q mutation i.e., VQVSS (SEQ ID NO: 38) or VTVQS (SEQ ID NO: 39), or combinations thereof.

In certain embodiments, polypeptides disclosed herein contains mutations at the C-terminal amino acid residues of framework 4 can be as follows: VTVKS (SEQ ID NO: 40), VTVQS (SEQ ID NO: 41), VKVSS (SEQ ID NO: 42) or VQVSS (SEQ ID NO: 43); or (ii) if a C-terminal extension is present: VTVKSX(n) (SEQ ID NO: 44), VTVQSX(n) (SEQ ID NO: 45), VKVSSX(n) (SEQ ID NO: 46) or VQVSSX(n) (SEQ ID NO: 47), such as VTVKSA (SEQ ID NO: 48), VTVQSA (SEQ ID NO: 49), VKVSSA (SEQ ID NO: 50) or VQVSSA (SEQ ID NO: 51).

In certain embodiments, polypeptides disclosed herein have a D or E at position 1 (i.e. an Q1D mutation). In certain embodiments, polypeptides disclosed herein have a C-terminal extension X(n) as described herein and has a D or E at position 1. Some specific, but non-limiting examples of other mutations/amino acid differences that may be present (i.e. compared to the sequences disclosed here) are: Q1D, Q1E, P41A, P41L, P41S, P4IT, P42E or T91A.

In certain embodiments, polypeptides disclosed herein are contemplated to contain a C-terminal extension such as a C-terminal alanine extension, i.e., an alanine residue at the C-terminal end of the ISVD-sequence providing a C-terminal sequence, e.g., VTVSSA (SEQ ID NO: 52).

In certain embodiments, the C-terminal end of polypeptides disclosed herein (or when they otherwise have an “exposed” C-terminal end in a protein, polypeptide or other compound or construct in which they are present, by which is generally meant that the C-terminal end of the ISV is not associated with or linked to a constant domain, such as a CHI domain); preferably also have a C-terminal extension of the formula (X)n, in which n is 1 to 10, preferably 1 to 5, such as 1, 2, 3, 4 or 5 (and preferably 1 or 2, such as 1); and each X is an (preferably naturally occurring) amino acid residue that is independently chosen from naturally occurring amino acid residues (although according to preferred one aspect, it does not comprise any cysteine residues), and preferably independently chosen from the group consisting of alanine (A), glycine (G), valine (V), leucine (L) or isoleucine (I).

In certain embodiments, such C-terminal extensions (X)n, X and n can be as follows: (a) n=1 and X=Ala; (b) n=2 and each X=Ala; (c) n=3 and each X=Ala; (d) n=2 and at least one X=Ala (with the remaining amino acid residue(s) X being independently chosen from any naturally occurring amino acid but preferably being independently chosen from Val, Leu and/or Ile); (e) n=3 and at least one X=Ala (with the remaining amino acid residue(s) X being independently chosen from any naturally occurring amino acid but preferably being independently chosen from Val, Leu and/or Ile); (f) n=3 and at least two X=Ala (with the remaining amino acid residue(s) X being independently chosen from any naturally occurring amino acid but preferably being independently chosen from Val, Leu and/or Ile); (g) n=1 and X=Gly; (h) n=2 and each X=Gly; (i) n=3 and each X=Gly, (j) n=2 and at least one X=Gly (with the remaining amino acid residue(s) X being independently chosen from any naturally occurring amino acid but preferably being independently chosen from Val, Leu and/or Ile); (k) n=3 and at least one X=Gly (with the remaining amino acid residue(s) X being independently chosen from any naturally occurring amino acid but preferably being independently chosen from Val, Leu and/or Ile); (1) n=3 and at least two X=Gly (with the remaining amino acid residue(s) X being independently chosen from any naturally occurring amino acid but preferably being independently chosen from Val, Leu and/or Ile); (m) n=2 and each X=Ala or Gly; (n) n=3 and each X=Ala or Gly; (o) n=3 and at least one X=Ala or Gly (with the remaining amino acid residue(s) X being independently chosen from any naturally occurring amino acid but preferably being independently chosen from Val, Leu and/or Ile); or (p) n=3 and at least two X=Ala or Gly (with the remaining amino acid residue(s) X being independently chosen from any naturally occurring amino acid but preferably being independently chosen from Val, Leu and/or Ile); with aspects (a), (b), (c), (g), (h), (i), (m) and (n) being particularly preferred, with aspects in which n=1 or 2 being preferred and aspects in which n=1 being particularly preferred.

In certain embodiments, any C-terminal extension does not contain a (free) cysteine residue (unless said cysteine residue is used or intended for further functionalization, for example for pegylation). In certain embodiments, examples of useful C-terminal extensions are the following amino acid sequences: A, AA, AAA, G, GG, GGG, AG, GA, AAG, AGG, AGA, GGA, GAA or GAG.

### Multimers or Immunoglobulin Single Variable Domain

In certain embodiments, this disclosure relates to polypeptides disclosed herein in the form of monomers, dimers, or multimers comprising immunoglobulin single variable domains reported herein. Dimers may be conjugated by a peptide linker or a disulfide bond from two cysteine amino acids within each of the two ISVDs. In certain embodiments, whether in the form of a monomer, multimer or dimer, the immunoglobulin single variable domains may contain a C terminal extension.

In certain embodiments, this disclosure relates to multimers or dimers comprising a first polypeptide and a second polypeptide, wherein each of said first and second polypeptide comprises at least one immunoglobulin single variable domain (ISVD) and a C-terminal extension. In certain embodiments, the polypeptide is a dimer connected by a linking group, e.g., AAA. In certain embodiments, the multimers or dimers comprise a cysteine residue (preferably at the C-terminus), wherein said first polypeptide and said second polypeptide are covalently linked via a disulfide bond between the cysteine moiety of said first polypeptide and the cysteine moiety of said second polypeptide.

In certain embodiments, this disclosure relates to method for making (polypeptide) dimers, comprising at least the steps of: (i) providing a first polypeptide, wherein said first polypeptide comprises at least one immunoglobulin single variable domain (ISVD) and a C-terminal extension comprising a cysteine moiety, preferably at the C-terminus; (ii) providing a second polypeptide, wherein said second polypeptide comprises at least one immunoglobulin single variable domain (ISVD) and a C-terminal extension comprising a cysteine moiety, preferably at the C-terminus; and (ill) oxidizing the thiol moiety of said cysteine moiety at the C-terminal extension, preferably at the C-terminus, of said first polypeptide and the thiol moiety of said cysteine moiety at the C-terminal extension, preferably at the C-terminus, of said second polypeptide to a disulfide derivative cystine; thereby making said dimers; and said disulfide derivative cystine is the only intermolecular disulfide bond present in the dimer.

Coupling of the polypeptides into a dimer can be performed by chemical conjugation in which the cysteines (preferably C-terminally located) in the C-terminal extension in each of said two polypeptides are oxidized to a disulfide derivative cystine via their thiol moieties at near neutral pH. In certain embodiments, the oxidation process is optimized by adding oxidizing copper ions (Cu²⁺), for instance in the form of CuSO₄. C-terminally located thiol moieties are oxidized after copper treatment.

In certain embodiments this disclosure relates to methods as described herein, wherein said first polypeptide and said second polypeptide are identical or are different. In certain embodiments, the polypeptide comprises at least one immunoglobulin single variable domain (ISVD) and further comprises one or more (preferably one) human proteins, e.g., serum albumin binding immunoglobulin single variable domain which may be on the C or N terminal, e.g., to prevent the binding of the “pre-existing antibodies.” See, e.g., U.S. Pat. No. 11,142,569.

### Methods of Use in Managing Blood Clotting Diseases or Conditions

In certain embodiments, this disclosure relates to methods of treating or preventing blood clotting comprising administering an effective amount of a polypeptide disclosed herein to a subject in need thereof. In certain embodiments, the subject is diagnosed with a disease or condition associated with abnormal blood clotting. In certain embodiments, disease or condition is thrombotic thrombocytopeniaurpura (TTP). In certain embodiments, subject is diagnosed with hereditary TTP, acquired idiopathic TTP, or autoantibodies to ADAMTS13.

In certain embodiments, this disclosure relates to methods of normalizing platelet counts, reducing the incidence of TTP-related death, recurrence of TTP, and other thromboembolic events comprising administering an effective amount of a polypeptide disclosed herein to a subject in need thereof.

In certain embodiments, this disclosure relates to methods comprising measuring the ability of VWF to aggregate platelets or bind recombinant GPIbα derived proteins in the presence of a polypeptide disclosed herein, comprising contacting the polypeptide, a sample comprising VWF and platelets, and thereafter measuring platelet aggregation.

In certain embodiments, this disclosure relates to methods comprising measuring VWF activity to diagnose bleeding disorders such as wherein the subject is exhibiting symptoms of, at risk of, or diagnosed with von Willebrand Disease (VWD), and clotting disorders such as micro thrombosis or TTP.

In certain embodiments, this disclosure relates to methods of treating or alleviating VWF-related diseases in a subject by administering to the subject a polypeptide comprising at least one ISVD against VWF A1 or an autoinhibitory module on the C terminal end or on the N terminal end of von Willebrand Factor (VWF) A1 domain, wherein the amount of the polypeptide administered is effective to reduce the time-to-response, to reduce exacerbations, to reduce hospitalization, to reduce ischemia, to reduce the death toll and/or to reduce the number of required Plasma Exchanges (PE).

In certain embodiments, this disclosure relates to methods of using specific dose ranges and dosing schedules for the polypeptides of the disclosure that result in one or more of these effects on VWF-related disease. In particular, the disclosure provides pharmacologically active agents, compositions, methods and/or dosing schedules that have certain advantages compared to the agents, compositions, methods and/or dosing schedules that are currently used and/or known in the art, including the requirement to less frequently give PE.

In certain embodiments, this disclosure relates to methods of using a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain for use in treating a VWF-related disease in a human in need thereof, comprising administering to said human a first dose of 1-80 mg, such as 5-40 mg, preferably 10 mg of said polypeptide.

In certain embodiments, this disclosure relates to methods of using a polypeptide as described herein, wherein said administering said polypeptide is followed within 5 min to 8 h by performing a first PE.

In certain embodiments, this disclosure relates to methods of using a polypeptide as described herein, wherein said administering of said first dose is preceded by performing a preceded Plasma Exchange (PE), preferably within 36 h, such as within 32 h, 30 h, 28 h, 26 h, 24 h, 22 h, 20 h, 18 h, 16 h, 14 h, 12 h, 10 h, 8 h, for instance within 7 h, 6 h, 5 h, 4 h, 3 h, 3 h, 1 h, 45 min, 30 min, 20 min, 15 min, 10 min or even 5 min of said first PE.

In certain embodiments, this disclosure relates to methods of using a polypeptide as described herein, wherein said treating a VWF-related disease in a human in need thereof, further comprises: (i) performing a PE; and (followed by) (ii) administering a dose of 1-80 mg, such as 5-40 mg of said polypeptide 5 min to 4 h after said PE of step (i); and (iii) optionally measuring the platelet count and/or ADAMTS13 activity of said patient, wherein step (i) and step (ii) are repeated once per day, preferably until the platelet count of said patient is greater than 150,000 platelets per microliter of blood and/or said ADAMTS13 activity is at least 10% such as at least 15%, 20%, 25%, 30%, 35%, 45% or even 50% of the ADAMTS13 reference activity.

In certain embodiments, this disclosure relates to methods of using a polypeptide as described herein, further comprising administering once per day a dose of 1-80 mg, such as 5-40 mg, preferably 10 mg of said polypeptide for at least 5, 10, 15, 20, 25, 30, 40, 50 60, 90 or even 120 days after the platelet count of said patient is greater than 150,000 platelets per microliter of blood for the first time.

In certain embodiments, this disclosure relates to methods of using a polypeptide as described herein, further comprising administering once per day a dose of 1-80 mg, such as 5-40 mg, preferably 10 mg of said polypeptide until said human enters remission.

In certain embodiments, this disclosure relates to methods of using a polypeptide as described herein, comprising administering said polypeptide until the ADAMTS13 activity is at least 10% such as at least 15%, 20%, 25%, 30%, 35%, 45% or even 50% of the ADAMTS13 reference activity.

In certain embodiments, this disclosure relates to methods of using a polypeptide as described herein, wherein said human suffers from an acute episode of TTP, an exacerbation of TTP or a relapse of TTP.

In certain embodiments, this disclosure relates to methods of using a polypeptide as described herein wherein said VWF-related disease is chosen from acute coronary syndrome (ACS), transient cerebral ischemic attack, unstable or stable angina pectoris, stroke, myocardial infarction, or thrombotic thrombocytopenia purpura (TTP).

In certain embodiments, this disclosure relates to methods of using a polypeptide as described herein for the treatment of a human patient susceptible to or diagnosed with a disease characterized by a VWF-related disease, comprising administering an effective amount of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain to the human patient.

In certain embodiments, this disclosure relates to methods of using a polypeptide as described herein for treating or preventing a VWF-related disease, such as TTP, comprising administering to a human, 1-80 mg, such as 5-40 mg, preferably 10 mg dose of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain, thereby reducing one or more symptoms associated with the VWF-related disease.

In certain embodiments, this disclosure relates to methods of using a polypeptide as described herein wherein said administering a polypeptide as described herein is followed within 5 min to 8 h, such as 15 min to 4 h by performing a first Plasma Exchange (PE).

In certain embodiments, this disclosure relates to methods of using a polypeptide as described herein wherein the risk of organ damage, ischemic damage and/or microthrombi formation is reduced by 10%, 20%, 30%, preferably by at least 40%, or even at least 50%, such as 60%, 70%, 80%, 90% or even to 100%; the risk of organ damage, ischemic damage and/or microthrombi formation is reduced by a factor 1.2. 1.3, 1.4, 1.5, 1.75, 2 or more, such as 3, 4, 5, 6, 7, 8, 9, or even 10, or even more such as 20, 50 or even 100; organ damage, ischemic damage and/or microthrombi formation is reduced preferably by at least 10%, 20%, 30%, 40%, or even at least 50%, such as 60%, 70%, 80%, 90% or even to 100%; organ damage, ischemic damage and/or microthrombi formation is reduced by a factor, 2 or more, such as 3, 4, 5, 6, 7, 8, 9, or even 10, or even more such as 20, 50 or even 100; organ damage markers, such as LDH levels, troponin T, troponin I levels, and/or creatinine levels, return to at least 40%, or even at least 50%, such as 60%, 70%, 80%, 90% or even to 100% of normal levels; organ damage markers, such as LDH levels, troponin T, troponin I levels, and/or creatinine levels, improve by at least 20%, such 30% or even higher, such as 40%, or even at least 50%, such as 60%, 70%, 80%, 90% or even to 100% of normal levels, Preferably, said organ damage, such as LDH levels, troponin T, troponin I levels, and/or creatinine levels, markers improve in less than 30 days of treatment, preferably, in less than 20 days of treatment, such as, less than 15, 10, 9, 8, 7, 6, 5, 4, 3, 2 days or even within 1 day; the number of platelets is kept at greater than 150000 platelets per microliter of blood; the risk of exacerbations is reduced by at least 10%, 20%, 30%, 40%, or even at least 50%, such as 60%, 70%, 80%, 90% or even to 100%; the risk of exacerbations is reduced by a factor, 2 or more, such as 3, 4, 5, 6, 7, 8, 9, or even 10, or even more such as 20, 50 or even 100; mortality due to said VWF related disease is reduced by 10%, 20%, 30%, preferably by at least 40%, or even at least 50%, such as 60%, 70%, 80%, 90% or even to 100%; mortality due to said VWF related disease is reduced by a factor 1.2, 1.3, 1.4, 1.5, 1.6, 1.75, 1.8, 2 or more, such as 3, 4, 5, 6, 7, 8, 9, or even 10, or even more such as 20, 50 or even 100.

In certain embodiments, this disclosure relates to methods of using a polypeptide as described herein comprising measuring the platelet number; and if said platelet number is lower than 150,000 platelets per microliter of blood, then repeating said polypeptide administration.

In certain embodiments, this disclosure relates to methods of using a polypeptide as described herein wherein administering the polypeptide is repeated until said platelet number is at least 150,000 platelets per microliter of blood on at least 2 consecutive measurements. Preferably, said 2 consecutive measurements are at least 24 h, more preferably 48 h apart, such as at least 3 days apart, or even more such as, 4, 5, 6, or even 7 days apart, preferable a week apart.

In certain embodiments, this disclosure relates to methods of using a polypeptide as described herein for reducing the risk of and/or preventing an acute episode of a VWF-related disease in a human in need thereof, comprising at least the following steps: (i) measuring the platelet number of said patient; and (ii) if said platelet number is lower than 150,000 platelets per microliter of blood then administering to said human a dose of 5-40 mg of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain; wherein administration of said polypeptide reduces the risk of and/or prevents an acute episode of a VWF-related disease.

In certain embodiments, this disclosure relates to methods of using a polypeptide as described herein for reducing the risk of and/or preventing ischemic damage, organ damage and/or microthrombi formation [causable by a VWF-related disease] in a human in need thereof, comprising at least the following step: (i) administering to said human a dose of 5-40 mg/day, preferably 10 mg/day of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain; wherein administration of said polypeptide reduces the risk of and/or prevents ischemic damage, organ damage and/or microthrombi formation by 10%, 20%, 30%, preferably by at least 40%, or even at least 50%, such as 60%, 70%, 80%, 90% or even to 100%. Preferably, administration of said polypeptide reduces the risk of and/or prevents ischemic damage, organ damage and/or microthrombi formation by a factor 1.2, 1.3, 1.4, 1.5, 1.6, 1.75, 1.8, 2 or more, such as 3, 4, 5, 6, 7, 8, 9, or even 10, or even more such as 20, 50 or even 100.

In certain embodiments, this disclosure relates to methods of using a polypeptide as described herein for inhibiting in a human the onset or progression of a VWF-related disease, such as TTP, the inhibition of which is effected by binding of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain, comprising administering to the human at a predefined interval effective inhibitory doses of said polypeptide, wherein each administration of the polypeptide delivers to the human from 0.1 mg per kg to 25 mg per kg of the human's body weight, so as to thereby inhibit the onset or progression of the disease in the human.

In certain embodiments, this disclosure relates to methods of using a polypeptide as described herein for reducing the likelihood of a human contracting ischemic organ damage by a VWF-related disease, which comprises administering to the human at a predefined dose a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain, wherein each administration of the antibody delivers to the human from 0.1 mg per kg to 25 mg per kg of the human's body weight, so as to thereby reduce the likelihood of the human contracting ischemic organ damage.

In certain embodiments, this disclosure relates to methods of treating or preventing a VWF-related disease, such as e.g. acute coronary syndrome (ACS), transient cerebral ischemic attack, unstable or stable angina pectoris, stroke, myocardial infarction or thrombotic thrombocytopenia purpura (TTP); said method comprising administering to a subject a pharmaceutical composition comprising the formulation as disclosed herein, thereby reducing one or more symptoms associated with said VWF-related disease. In particular, said VWF-related disease is TTP.

In certain embodiments, this disclosure relates to methods for the treatment of a human patient susceptible to or diagnosed with a disease characterized by a VWF-related disease, comprising administering an effective amount of a polypeptide comprising at least one immunoglobulin single variable domain (ISVD) against von Willebrand Factor (VWF) A1 or an autoinhibitory module on the C terminal end or on the N terminal end of von Willebrand Factor (VWF) A1 domain to the human patient.

In certain embodiments, this disclosure relates to methods of treating or preventing spontaneous bleeding in a subject comprising administering an effective amount of a polypeptide comprising a ISVD disclosed herein wherein the ISVD is a platelet aggregation activator. In certain embodiments, this disclosure relates to methods of treating or preventing spontaneous bleeding comprising administering an effective amount of a platelet clotting activating polypeptide disclosed herein to a subject in need thereof.

In certain embodiments, the subject is exhibiting symptoms of, at risk of, or diagnosed with von Willebrand Disease (VWD), thrombocytopenia, or hemophilia.

In certain embodiments, thrombocytopenia due to a hematological disorder or malignancy such as chronic immune thrombocytopenia, aplastic anemia, or a hematological malignancy such as leukemia, acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL), lymphoma, Hodgkin's lymphoma, or Non-Hodgkin's lymphoma.

In certain embodiments, this disclosure relates to methods of treating or preventing blood clotting or thrombosis comprising administering an effective amount of a polypeptide of the disclosure to a subject in need thereof. In certain embodiments the subject is exposed to a mechanical circulatory support system, e.g., a device that provides extracorporeal membrane oxygenation (ECMO).

### Methods of Use in Assays

Von Willebrand's disease (vWD) arises from quantitative and qualitative abnormalities in VWF. It is the most common inherited bleeding disorder. Three major categories of vWD are distinguished (Sadler, Thromb Haemost 1994; 71:520-5). Types 1 and 3 refer to mild and severe quantitative deficiency of VWF respectively, whereas type 2 refers to qualitative abnormalities. Qualitative type 2 vWD is further divided into four subtypes (A, B, M and N). In type 2A individuals, there is an absence of high molecular weight (HMW) multimers. Type 2B variants show an increased affinity for glycoprotein GP1b-α on platelets resulting in a loss of HMW multimers in plasma. VWD type 2M includes variants in which platelet adhesion is impaired but the VWF multimer distribution is normal.

Screening tests used in order to evaluate a new patient suspected suffering from vWD are: bleeding time (BT), VWF antigen (VWF: Ag) level and VWF ristocetin-induced platelet agglutination, termed ristocetin cofactor activity (VWF: RCo). Measurement of the VWF: Ag is most frequently performed as an ELISA assay.

The ristocetin cofactor assay is usually carried out by mixing a plasma sample of a subject with platelets fixed with formaldehyde and ristocetin (See U.S. Published Patent App. No. 2010/0099202. Ristocetin induces binding of the VWF present in the sample to the GP1b-α receptor of the added platelets, resulting in the latter aggregating. The extent of this aggregation reaction correlates with the amount of active VWF present in the sample. Said aggregation reaction may be recorded optically, for example, by measuring the increase in transmission, thereby enabling the VWF: RCo activity to be quantified.

Compared to a VWF antigen assay, the ristocetin cofactor assay has the advantage of determining the activity of VWF and therefore enables functional VWF disorders and the classification of various subtypes of von Willebrand syndrome, only some of which are also accompanied by a reduced VWF antigen concentration, to be recognized. Said subtypes are often classified by forming the ratio of VWF ristocetin cofactor activity (VWF: RCo) and VWF antigen concentration (VWF: Ag). A VWF: RCo/VWF: Ag ratio of less than 1 is characteristic for the von Willebrand syndrome subtypes 2A, 2B and 2M. A frequently recommended threshold is a ratio of 0.7.

In certain embodiments, this disclosure relates to methods and kits for measuring von Willebrand factor (VWF), and more particularly to methods and kits for measuring VWF. In certain embodiments, ristocetin is replaced with a polypeptide/ISVD antibody platelet aggregation activator reported herein.

In certain embodiments, this disclosure relates to methods of using polypeptides comprising ISVDs or variants thereof disclosed herein optionally conjugated to a label that bind, inhibit and/or activate VWF in assays. In certain embodiments, methods comprise mixing polypeptides disclosed herein that bind VWF A1 or an autoinhibitory module on the C terminal end or on the N terminal end of von Willebrand Factor (VWF) A1 domain with a blood product and measuring the amount of VWF present in blood.

In certain embodiments, methods comprise mixing a blood product, e.g., platelets and plasma (patient plasma or platelets or fixed platelets or standard plasma), from a subject and determining the amount of VWF in the blood product and/or the ability to induce platelet aggregation.

In certain embodiments, this disclosure relates to a solid surface comprising a VWF A1 or an autoinhibitory module on the C terminal end or on the N terminal end of von Willebrand Factor (VWF) A1 domain binding polypeptide disclosed herein immobilized to the solid surface, e.g., beads (magnetic) coated with the polypeptide, and using such in a solid surface in the assay. In certain embodiments, assays include using a VWF A1 or an autoinhibitory module on the C terminal end or on the N terminal end of von Willebrand Factor (VWF) A1 domain binding polypeptide disclosed herein wherein GPIbα or fragment is bound to a solid surface, e.g., ELISA platform, beads, etc. In certain embodiments, measuring binding may be with and without a VWF A1 or an autoinhibitory module on the C terminal end or on the N terminal end of von Willebrand Factor (VWF) A1 domain binding polypeptide disclosed herein providing a comparison.

In certain embodiments, methods comprise obtaining a blood product, e.g. platelets and plasma (patient plasma or platelets or fixed platelets or standard plasma), from a subject and mixing the blood product with platelets in the presence of a VWF A1 or an autoinhibitory module on the C terminal end or on the N terminal end of von Willebrand Factor (VWF) A1 domain binding polypeptide disclosed herein and measuring the ability of a binding polypeptide disclosed herein to induce or inhibit platelet aggregation, e.g., VWF aggregates (washed) or agglutinates (lyophilized or paraformaldehyde-fixed) platelets in the presence and/or absence of VWF binding polypeptide disclosed herein.

In certain embodiments, methods comprise using a VWF binding polypeptide disclosed herein to evaluate the glycoprotein 1bα (GP1bα)-mediated binding role of VWF in a qualitative test for VWF. In certain embodiments, aggregation is detected by aggregometer or by visual agglutination or turbidity.

In certain embodiments, the polypeptide comprising an immunoglobulin single variable domain that specifically binds A1 or the autoinhibitory module on the N terminal end of the A1 domain in von Willebrand Factor (VWF) have complementary determining region (CDR) 1, CDR 2, and CDR 3 and framework regions (FRs), and wherein the CDR1, CDR 2, and CDR 3 are from camelid antibody Cd1C4, Cd1C11, Cd1D12 or variants thereof.

In certain embodiments, this disclosure relates to methods of measuring the ability of VWF to aggregate platelets in the presence of a polypeptide disclosed herein, comprising contacting the polypeptide, a sample comprising VWF and platelets, and thereafter measuring platelet aggregation.

In certain embodiments, the sample comprising VWF and platelets is a blood sample from a subject at risk of, exhibiting symptoms of, or diagnosed with von Willebrand Disease (VWD) or thrombocytopenia.

In certain embodiments, platelets are platelet rich plasma or normal plasma of a subject.

In certain embodiments, symptoms of thrombocytopenia are excessive bruising, superficial bleeding into the skin, petechiae on the lower legs, abnormal prolonged bleeding in mouth, nose, from cuts, or combinations thereof.

In certain embodiments, the subject is diagnosing with type 2B von Willebrand Disease (VWD) when platelet aggregation is increased compared to measuring platelet aggregation after contacting the sample comprising VWF and platelets of the subject without the polypeptide.

### Pharmaceutical Compositions

In certain embodiments, this disclosure contemplates pharmaceutical compositions comprising ISVSs or polypeptide disclosed herein, and optionally at least one pharmaceutically acceptable carrier, diluent, or excipient. Any pharmaceutical product or composition comprising any ISVDs (polypeptides disclosed herein) may also comprise one or more further components known per se for use in pharmaceutical products or compositions (i.e. depending on the intended pharmaceutical form) and/or for example one or more other compounds or active principles intended for therapeutic use (i.e. to provide a combination product). The pharmaceutical compositions can be administered in any suitable manner that allows the compound or polypeptide to enter the circulation, such as intravenously, via injection or infusion, or in any other suitable manner (including oral administration, subcutaneous administration, intramuscular administration, administration through the skin, intranasal administration, administration via the lungs, etc.).

In certain embodiments, this disclosure contemplates a polypeptide formulation as described herein, wherein said formulation comprises a citrate or phosphate buffer with a pH in the range of 5.0 to 7.5.

In certain embodiments, this disclosure contemplates a polypeptide formulation as described herein, wherein said formulation is in liquid, lyophilized, spray-dried, reconstituted lyophilized or frozen form.

In certain embodiments, this disclosure contemplates a formulation comprising: (a) an ISVD antibody disclosed herein at a concentration from about 0.1 mg/mL to about 80 mg/mL; (b) an excipient chosen from sucrose, glycine, mannitol, trehalose or NaCl at a concentration of about 1% to about 15% (w/v); (c) Tween-80 at a concentration of about 0.001% to 0.5% (v/v); and (d) a buffer chosen from citrate buffer at a concentration of about 5 mM to about 200 mM such that the pH of the formulation is about 6.0 to 7.0 and a phosphate buffer at a concentration of about 10 mM to about 50 mM such that the pH of the formulation is about 6.5 to 7.5, for use in treating a VWF-related disease in a human in need thereof, by administering to the human a 1-80 mg, such as 5-40 mg dose, preferably 10 mg of said polypeptide, wherein said dose is followed within 5 min to 8 h, such as 15 min to 4 h by a first Plasma Exchange (PE).

In certain embodiments, this disclosure contemplates a pharmaceutical unit dosage form suitable for parenteral administration to a patient, preferably a human patient, comprising a polypeptide as described herein or a formulation as described herein.

The term “pharmaceutical formulation” refers to a preparation which is in such form as to permit the biological activity of the active ingredient (the polypeptide) to be effective, and which contains no additional components which are unacceptably toxic to a subject to which the formulation would be administered. Such formulations are sterile, “Pharmaceutically acceptable” excipients (vehicles, additives) are those which can reasonably be administered to a subject mammal to provide an effective dose of the active ingredient employed.

The term “excipient” as used herein refers to an inert substance which is commonly used as a diluent, vehicle, preservative, surfactant, binder, carrier, or stabilizing agent for compounds which impart a beneficial physical property to a formulation. The skilled person is familiar with excipients suitable for pharmaceutical purposes, which may have particular functions in the formulation, such as stabilization, preservation, etc.

A “sterile” formulation is aseptic or free or essentially free from all living microorganisms and their spores. This is readily accomplished by filtration through sterile filtration membranes.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. The formulation comprises an aqueous carrier. The aqueous carrier is in particular a buffer.

As used herein, “buffer” refers to a buffered solution that resists changes in pH by the action of its acid-base conjugate components. The formulation of the invention comprises a buffer selected from at least one of citrate or phosphate buffer, preferably a citrate buffer. Buffers enhance the stability of the VWF binders. The pH of the formulation is typically in the range 5.0 to 7.5, wherein each value is understood to encompass a range of plus or minus 0.2. The most advantageous pH will depend on the buffer comprised in the formulation. Hence, a formulation comprising a phosphate buffer, which preferably has a pH in the range of 6.5 to 7.5, preferably 6.9, 7.0, 7.1, e.g. 7.1. A formulation comprising a citrate buffer is suitable for storage and use. A formulation comprising a citrate buffer, which preferably has a pH between 6.0 and 7.0, more preferably 6.1, 6.2, 6.3, 6.4, 6.5, 6.6, 6.7, 6.8 or 6.9, e.g., 6.5.

In certain embodiments, formulations comprise the polypeptides with the immunoglobulin single variable domains against VWF A1 or an autoinhibitory module on the C terminal end or on the N terminal end of von Willebrand Factor (VWF) A1 domain at a concentration that is suitable for clinical purposes, which includes concentrations used in stock solutions for dilution prior to use on the patient. Typical concentrations comprise the non-limiting examples of concentrations in the range of 0.1 to 150 mg/mL, such as 1-100 mg/mL, 5-80 mg/mL, or 10-40 mg/mL, preferably 10 mg/mL, wherein each value is understood to optionally encompass a range (e.g., a value of 10 optionally encompasses a range of 8 to 12 mg/mL).

In a further embodiment, the formulation may further comprise a detergent or surfactant. A “surfactant” refers to a surface-active agent, preferably a nonionic surfactant. Examples of surfactants herein include polysorbate; poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; polyethyl glycol, polypropyl glycol, and copolymers of ethylene and propylene glycol etc.

The formulation may further comprise excipients such as preservatives. A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. In one embodiment, the preservative herein is benzyl alcohol.

The formulation may further comprise stabilizing agents, such as a polyols. A “polyol” is a substance with multiple hydroxyl groups, and includes sugars (reducing and nonreducing sugars), sugar alcohols and sugar acids, A polyol may optionally be included in the formulation, for instance to improve stability. In certain embodiments, polyols herein have a molecular weight which is less than about 600 kD (e.g. in the range from about 120 to about 400 kD). A “reducing sugar” is one which contains a hemi-acetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, and raffinose. Mannitol, xylitol, erythritol, threitol, sorbitol and glycerol are examples of sugar alcohols. As to sugar acids, these include L-gluconate and metallic salts thereof. Where it desired that the formulation is freeze-thaw stable, the polyol is preferably one which does not crystallize at freezing temperatures (e.g. −20 degrees C.) such that it destabilizes the peptide in the formulation. In certain embodiments, nonreducing sugars such as sucrose and trehalose are examples of polyols, with sucrose being preferred, despite the solution stability of trehalose.

Commonly used stabilizers and preservatives are well known to the skilled person (see e.g. WO2010/077422). Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, hydrophilic polymers such as polyvinyl pyrrolidone, cellulose based substances, polyethylene glycol, sodium carboxymethylcellulose, polyacrylates, waxes, gelatin, polyethylene polyoxypropylene block polymers, polyethylene glycol and antioxidants including ascorbic acid and methionine; preservatives; low molecular weight (less than about 10 residues) polypeptides; proteins; and amino acids such as glycine, glutamine, asparagine, histidine, arginine, or lysine. In advantageous embodiments, the excipient may be one or more selected from the list consisting of NaCl, trehalose, sucrose, mannitol, or glycine.

The disclosure also encompasses products obtainable by further processing of a liquid formulation, such as a frozen, lyophilized or spray-dried product. Upon reconstitution, these solid products can become liquid formulations as described herein (but are not limited thereto). In its broadest sense, therefore, the term “formulation” encompasses both liquid and solid formulations. However, solid formulations are understood as derivable from the liquid formulations (e.g. by freezing, freeze-drying or spray-drying), and hence have various characteristics that are defined by the features specified for liquid formulations herein. The invention does not exclude reconstitution that leads to a composition that deviates from the original composition before e.g. freeze- or spray drying. accordingly, the lyophilized formulation may be reconstituted to produce a formulation that has a concentration that differs from the original concentration (i.e., before lyophilization), depending upon the amount of water or diluent added to the lyophilate relative to the volume of liquid that was originally freeze-dried.

In a preferred embodiment, the formulations are isotonic in relation to human blood. Isotonic solutions possess the same osmotic pressure as blood plasma, and so can be intravenously infused into a subject without changing the osmotic pressure of the subject's blood plasma. Tonicity can be expressed in terms of osmolality, which can be a theoretical osmolality, or preferably an experimentally determined osmolality.

### Kits

In certain embodiments, this disclosure relates to kits containing materials useful for the treatment of a disease as described above is provided. In certain embodiments, the kit comprises a container, a product label and a package insert. Suitable containers include, for example, bottles, vials, syringes, etc. The containers may be of a variety of materials such as glass or plastic. The container holds the composition which is effective in treating the condition and may have a sterile access port (for example the container may be an intravenous solution bag or a vial having a stopper pierceable by a hypodermic injection needle). At least one active agent in the composition is the polypeptide disclosed herein. The product label on, or associated with, the container indicates that the composition is used for treating the condition of choice. In certain embodiments, the kit may further comprise a second container comprising a pharmaceutically acceptable buffer, such as a phosphate buffer saline or a citrate buffered saline as described herein. It may further include other materials desirable from a user or commercial standpoint, including other buffers, diluents, filters, needles, and syringes. In certain embodiments, a dosage unit form can be e.g., in the format of a prefilled syringe, an ampoule, cartridge or a vial.

In certain embodiments, this disclosure relates to kits or articles of manufacture, comprising a polypeptide or the formulation thereof as disclosed herein and instructions for use by, e.g., a healthcare professional. The kits or articles of manufacture may include a vial or a syringe containing the formulation as described herein.

Preferably, the vial or syringe is composed of glass, plastic, or a polymeric material chosen from a cyclic olefin polymer or copolymer. The syringe, ampoule, cartridge, or vial can be manufactured of any suitable material, such as glass or plastic and may include rubber materials, such as rubber stoppers for vials and rubber plungers and rubber seals for syringes and cartridges.

In certain embodiments, the kit may further comprise instructions for use and/or a clinical package leaflet. In any embodiment of the products as defined herein, this disclosure also encompasses the presence of packaging material, instructions for use, and/or clinical package leaflets, e.g., as required by regulatory aspects.

Immunoglobulin Single Variable Domain Antibodies that Target and Inhibit Human Von Willebrand Factor

This disclosure relates to polypeptides/ISVDs and variants useful as therapeutics to treat diseases that are mediated by over-active von Willebrand factor (VWF). In many inflammatory conditions, activated VWF causes thrombosis in capillary vessels and platelet depletion in other tissues.

Caplacizumab (WO 2011/067160) is a humanized ISVD antibody that targets VWF and inhibits its interaction with GPIbα having the following sequence:

(SEQ ID NO: 53)
EVQLVESGGGLVQPGGSLRLSCAASGRITSYNPMGWFRQAPGKGRELVA
AISRTGGSTYYPDSVEGRFTISRDNAKRMVYLQMNSLRAEDTAVYYCAA
AGVRAEDGRVRTLPSEYTFWGQGTQVTVSSAAAEVQLVESGGGLVQPGG
SLRLSCAASGRTFSYNPMGWFRQAPGKGRELVAAISRTGGSTYYPDSVE
GRFTISRDNAKRMVYLQMNSLRAEDTAVYYCAAAGVRAEDGRVRTLPSE
YTFWGQGTQVTVSS.

It has been discovered that caplacizumab inhibits VWF binding to GPIbα by binding to a regulatory element called the autoinhibitory module (AIM) that is located around the A1 domain. Thus, caplacizumab is an indirect inhibitor of VWF. Through mechanism-based screenings new llama ISVDs have been identified that bind to A1 or the AIM and indirectly inhibit human VWF like caplacizumab. Thus, this disclosure relates to these ISVDs, humanized modifications, and uses in treating VWF-mediated conditions.

Von Willebrand factor (VWF) is primarily secreted from endothelial cells lining the blood vessel, and it mediates hemostasis, thrombosis, and thromboinflammation by sensing and responding to changes in blood shear flow. Under low shear conditions, plasma VWF is autoinhibited and does not bind glycoprotein (GP)Ibα, the major subunit of the platelet GPIb-IX complex. However, when VWF is either exposed to elevated shear or immobilized under flow, it experiences tension and subsequently exposes its A1 domain for binding to GPIbα and platelets. The binding transmits a signal into platelets that leads to aggregation and clearance. Abnormal pathological binding of VWF to platelets in circulation leads to microthrombosis, thrombotic thrombocytopenia, and sometimes organ failure.

Under several conditions independent of flow change, VWF can overcome its autoinhibition and bind to GPIbα with high affinity. These conditions are present in some disease states, the most notable of which is type 2B von Willebrand disease (vWD) associated with mutations located in or flanking regions around A1 suggesting that autoinhibitory elements are localized around A1.

Glycopeptide ristocetin and snake venom protein, botrocetin, induce VWF binding to GPIbα in the absence of shear. Ristocetin, but not botrocetin, mimics shear dependent activation of VWF. Although ristocetin is widely used in research and diagnostic tests, and the ristocetin-binding site in VWF has been mapped to include a proline-rich sequence following A1.

Crystal structures of individual domains of VWF show that the D′D3 assembly extends to residue 1237 and that the A2 domain starts at residue 1494. The A1 domain is encompassed by the 1272-1458 disulfide bond and flanked by stretches of sequences (residues 1238-1271 and 1459-1493) that are O-glycosylated (FIG. 1A). Truncating these flanking regions around the disulfide bond has yielded A1 fragments with disparate affinities for GPIbα. Fragments of A1 with differential affinities for GPIbα suggests that both N- and C-terminal flanking sequences interact cooperatively and thus may constitute an autoinhibitory module (AIM). Experiments indicate that discontinuous AIM does resist tensile force, and cooperatively unfolds above a certain threshold of force to expose A1. Experiments indicate disruption of the AIM increases A1 affinity for GPIbα under pathologically relevant conditions.

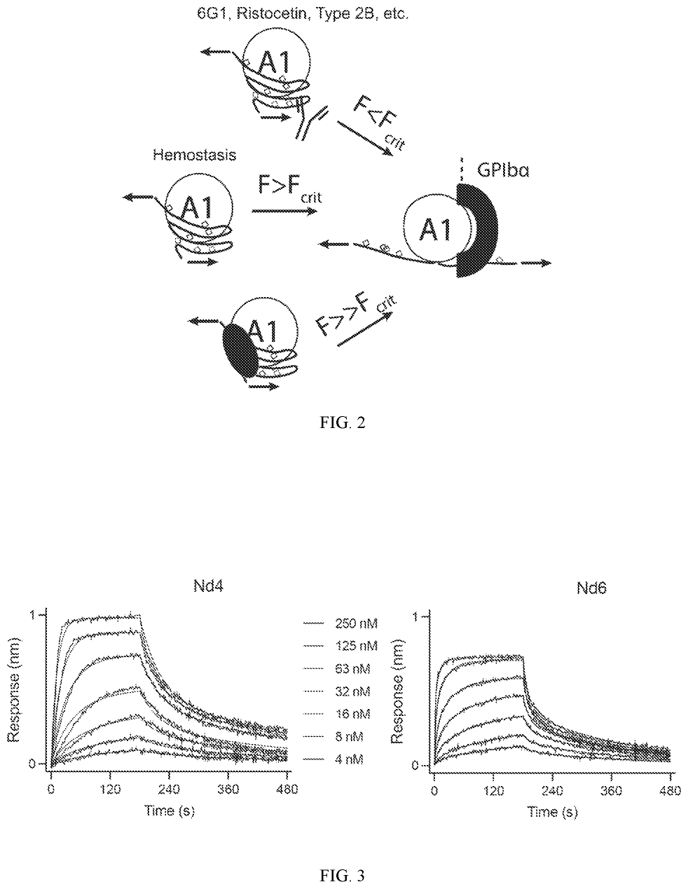

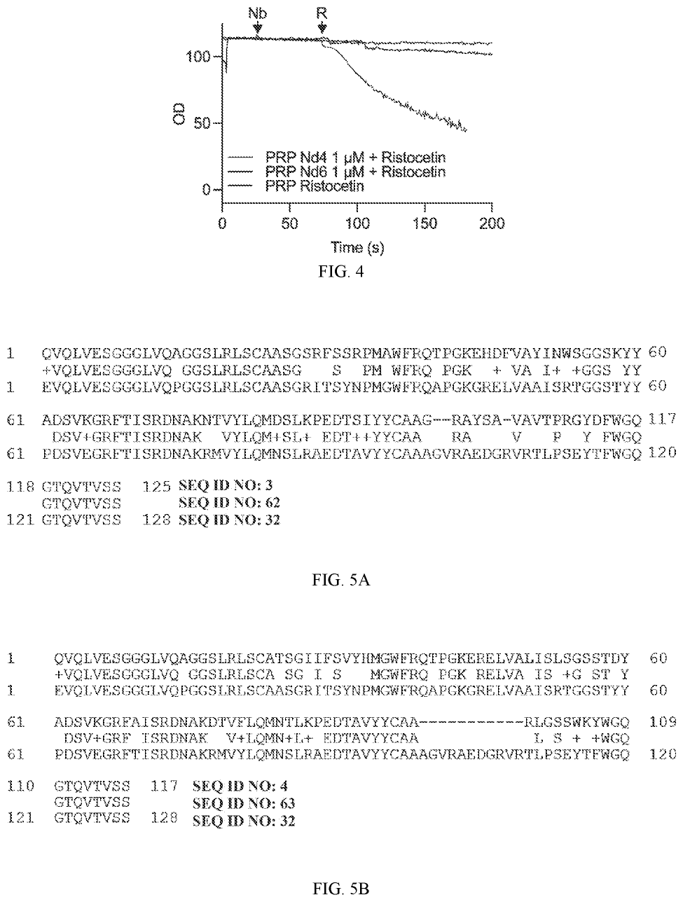

Coupling structural, functional, and single-molecule analysis there is evidence for a cooperative mechanical modulation of A1 binding by both halves of the discontinuous AIM. Deletion of either half of the AIM, the introduction of a type 2B VWD mutation at the AIM/A1 interface, or addition of a ristocetin-mimicking antibody that binds to C terminal (to A1) AIM residues (CAIM) results in the significantly decreased mechanical stability of the AIM and drastically increased activity of A1. These results suggest that widely documented factors of VWF activation, such as shear force, type 2B VWD mutations, and ristocetin, may share a common molecular mechanism—by destabilizing or disrupting the AIM that shields the A1 domain (FIG. 2).

Experiments indicate AIM constitutes a single structural unit as it unfolds under tensile force mostly in a single extension event instead of separate unfolding events of NAIM and CAIM. The measured contour length increase of 26.6 nm corresponds to about 67 residues present in unstructured regions after the unfolding event, which most likely include both NAIM and CAIM. The unfolding force of the AIM is greater than the individual unfolding forces of NAIM and CAIM, providing additional evidence supporting the cooperativity of NAIM and CAIM. Moreover, type 2B mutations or addition of the ristocetin-like antibody also disrupted cooperative unfolding of the AIM, resulting in significantly lowered unfolding force and shortened contour length increase.

The interaction of platelet GPIbα with VWF through their respective ligand-binding domain and A1 domains is critical to thrombus formation in many thrombotic diseases. Since the GPIbα-VWF interaction is essential to primary hemostasis, as genetic deletion of either protein would result in a severe bleeding disorder, pharmacological inhibition of the interaction may lead to side effects of severe bleeding. Caplacizumab is an inhibitor of the GPIbα-VWF interaction that has been clinically approved. TTP patients treated with caplacizumab showed a small risk of a bleeding event, mostly limited to epistaxis or gingival bleeding. The severity of these events was low and almost entirely resolved without intervention.

Caplacizumab is composed of two copies of the ISVD PMP12A2h1 (designated as VHH81) linked by a tri-alanine sequence. Monomeric recombinant VHH81 was produced in bacteria, and it bound purified VWF and plasma-derived VWF with an affinity of about 20-nM. VHH81 dose-dependently inhibited ristocetin-induced binding of AIM-A1 to platelet GPIb-IX and platelet aggregation VHH81 differs from all the previously reported inhibitors of the GPIbα-VWF interaction. Experiments indicate that it does not directly interfere with the GPIbα-binding site in A1 but rather binds to primarily NAIM residues. VHH81 stabilizes the AIM-A1 interface, as exemplified by the interaction between residues 1341 and 1264 and increases the unfolding force threshold for the AIM. The binding of VHH81 raises the shear threshold of VWF mechanoactivation (FIG. 2). These results could explain why at very high shear rates VHH81 could not completely abolish VWF-mediated platelet adhesion, whereas traditional antagonists could and thus would render VWF completely incapable of platelet capture at high shear rates as required for normal hemostasis. This important difference may help explain the lack of major bleeding risk with caplacizumab. The approach of targeting the AIM may be more productive with less impact on hemostasis than that of direct antagonism of the A1 domain.

### Construction and Production of ISVDs

The sequence of VHH81 was obtained from international patent WO2011/067160 (clone PMP12A2h1) and its encoding DNA fragment was synthesized by Integrated DNA Technologies. For crystallization experiments, primers were used to amplify a gene fragment encoding VHH81 with C-terminal hexahistidine (VHH81-6His), cloned into a modified pDEST14 vector, and produced in the cytoplasm of SHuffle™ T7 Express cells. To induce expression in both cases, 0.4 mM IPTG was added to bacteria culture at OD600 of 0.9 at 30° C. After 4-5 h, cells were centrifuged at 8,000 g for 20 min at room temperature and lysed with BugBuster™ with benzonase (Novagen/Sigma-Aldrich) according to the directions of the manufacturer. The lysate was centrifuged at 17,000 g and supernatant filtered by a Steriflip™ unit (Millipore). VHH81-6His was purified by Ni-affinity chromatography and gel filtration chromatography in PBS. Purified protein was flash-frozen in liquid nitrogen and stored at −80° C. until use.

To express Flag-VHH81 for BLI experiments, the gene fragment encoding VHH81 with an N-terminal FLAG™ tag, a C-terminal TEV protease cleavage sequence, and a 6His tag was cloned into the pET22b+plasmid and expressed in SHuffle™ T7 Express cells (New England Biolabs). To purify Flag-VHH81 from the periplasm, cell pellets were resuspended in 30 mM Tris-HCl, 20% sucrose, pH 8.0, at 80 ml per gram wet weight. EDTA was added dropwise to 1 mM. Cells were incubated on ice for 10 min with gentle agitation. The cell suspension was centrifuged at 8000 g for 20 min at 4° C., and the pellet resuspended in the same volume of ice-cold 5 mM MgSO₄. The cell suspension was incubated on ice for 10 min with gentle agitation. The suspension was centrifuged as before, and the protein in the supernatant was purified by Ni-affinity chromatography. Cleavage of the 6His tag by recombinant TEV protease (1 mg ISVD/125 μg protease) was performed overnight at 4° C. in PBS with 10% glycerol. The mixture was applied to a His-Trap column and the flow-through containing Flag-VHH81 collected and analyzed via western blot and ELISA. The lack of the 6His tag in purified Flag-VHH81 was verified via immunoblot with anti-His antibody 4E3D10H2/E3 at 1:2000 dilution or ELISA.

### Recombinant VWF and BioSpy-VWF Fragments

For recombinant VWF fragments e.g., 1268-1493 (A1-CAIM), the encoding DNA fragment was amplified from the expression vector encoding 1238-1493 10 His using primers, and subcloned into the pcDNA3.1-Hygro(+) vector (Invitrogen) as an XbaI-NheI fragment. The resulting vector was subsequently transfected into Expi293F cells for stable protein expression and purification as described using a GE Healthcare Ni Sepharose™ excel column followed by size exclusion chromatography on a GE Healthcare HiLoad 16/600 Superdex™ 200 pg column.

To clone BioSpy-VWF constructs, a decahistidine (10His) and SpyTag™ AHIVMVDAYKPTK (SEQ ID NO: 54) sequence was appended to the C-terminus of VWF fragments using primers. Each gene fragment was ligated into a pBIG4a vector using SpeI and XhoI sites such that a consensus Kozak sequence, al-antitrypsin signal sequence, and a BioTag™ was appended to the N terminus. The expression cassette was subsequently subcloned into pcDNA3.1-Hygro as a NheI-XhoI fragment. Type 2B constructs were generated by site directed mutagenesis using primers.

Each pcDNA-BioSpy-VWF vector was transfected into adherent Expi293F-BirA cells using Lipofectamine™ 3000. Single clones were selected using 250 μg/mL hygromycin B (ThermoFisher).

### Immunoglobulin Single Variable Domain (ISVD) Antibody Inhibitors Von Willebrand Factor

Activated VWF binds to the ligand-binding domain (LBD) of platelet GPIbα receptor and activates the platelet. VWF binds to platelet GPIbα through its A1 domain. In plasma VWF, the A1 domain is delimited by the 1272-1458 disulfide bond, and its GPIbα-binding site is partially masked by the autoinhibitory module (AIM). As illustrated in FIG. 2, the AIM consists of two stretches of sequences around A1, which together cooperatively form a meso-stable structural module. The AIM can be disrupted by a number of factors, including type 2B vWD mutations, ristocetin, and a ristocetin-mimicking antibody 6G1. In addition, the AIM can be stabilized by caplacizumab, which binds to a sequence in the N-terminal part of the AIM (NAIM). Thus, AIM may be targeted for indirect inhibition of VWF activity.

To identify inhibitors of human VWF, a recombinant protein containing human VWF residues 1238-1481 was generated from transfected human embryonic kidney cells. The protein was then injected into a llama. Once the animal was confirmed to have immune response to the protein, peripheral blood mononuclear cells were isolated, and cDNA was prepared and amplified. The ISVDs genes in the cDNA were subcloned into pYDNB, a derivative of the pCTcon2 plasmid. The ISVDs were expressed in a yeast display library. The library was screened against recombinant proteins containing human VWF residues 1238-1461 (NAIM-A1) or 1268-1493 (A1-CAIM), both of which contain activated A1 domain. The constructs that bound to 1238-1461 were depleted from the library, and the remaining population should only contain binders to residues 1462-1481 (FIGS. 1A and 1B). Likewise, the constructs that bound to 1268-1493 were depleted and the remaining population should contain binders to residues 1238-1267.

The depleted library subsets were then screened for the following properties:
binding to recombinant protein containing human VWF residues 1238-1493 (i.e. full sequence of AIM-A1)
blocking binding of recombinant AIM-A1 to the N-terminal ligand-binding domain of human GPIbα that bears a platelet-type VWF mutation.

Four clones, R12CdB9, R12NdB2, R6Nd4, and R6Nd6 have been identified. The protein sequences with C terminal HIS tags are listed below.

R12CdB9 sequence:
(SEQ ID NO: 55)
MQVQLVESGG GLVQAGGSLR LSCAASGLTF MDHVMGWFRQ
APGKEREFVA AVGRSAIMRD YADLVKGRFT ISRDNAKNTV
YLQMDSLKFE DTAVYYCAAR TPFPSDMTWS LPNDYIYWGQ
GAQVTVSSEP KTPKPQPLEH HHHHH
R12NdB2 sequence:
(SEQ ID NO: 56)
MQVQLVESGG GLVQAGGSLR LSCAASGEKL TQYVVGWLRQ
APGKEREFVA SISRSGVFTN YADSVKGRFT TSKDNAKNTV
YLQMNSLTPD DTAIYFCAAD SRYSGTDWRV RGEYWGQGTQ
VTVSSEPKTP KPQPLEHHHH HH
R6Nd4 sequence:
(SEQ ID NO: 57)
MQVQLVESGG GLVQAGGSLR LSCAASGSRF SSRPMAWFRQ
TPGKEHDFVA YINWSGGSKY YADSVKGRFT ISRDNAKNTV
YLQMDSLKPE DTSIYYCAAG RAYSAVAVTP RGYDFWGQGT
QVTVSSEPKT PKPQPLEHHH HHH
R6Nd6 sequence:
(SEQ ID NO: 58)
MQVQLVESGG GLVQAGGSLR LSCATSGIIF SVYHMGWFRQ
TPGKERELVA LISLSGSSTD YADSVKGRFA ISRDNAKDTV
FLQMNTLKPE DTAVYYCAAR LGSSWKYWGQ GTQVTVSSEP
KTPKPQPLEH HHHHH

Since R12CdB9 is identified from the aforementioned negative screen against A1-CAIM, its epitope is expected to be located in NAIM (i.e., residues 1238-1267). Likewise, R12NdB2, R6Nd4, R6Nd6 are identified from negative screen against NAIM-A1, their epitopes are expected to be located in CAIM (FIG. 1A). Each ISVDs protein has been subcloned into an expression vector and produced from E. coli with a C-terminal His tag.

ISVDs R6Nd4 and R6Nd6 bind to human VWF AIM-A1 protein with nanomolar affinity (FIG. 3). Addition of ISVDs R6Nd4 and R6Nd6 inhibit ristocetin induced platelet aggregation. Ristocetin is known to activate platelets and induce platelet aggregation in a VWF dependent manner, which can be completely inhibited by inhibitors of the VWF-GPIbα interaction.

Platelet adhesion on a collagen coated surface under flow shear is a commonly used assay to evaluate platelet function. In this assay, human whole blood is pumped through a microfluidic chamber and fluorescently labeled platelets are monitored for binding to collagen over time. Under high shear rate (e.g., 3000/s), VWF attached to coated collagen becomes activated to mediate platelet adhesion. Direct inhibitors of VWF such as ARC1179 completely blocks platelet adhesion under high shear, whereas caplacizumab reduces platelet adhesion but does not completely block platelet adhesion. Like caplacizumab, both R6Nd4 and R6Nd6 show a modest reduction in platelet adhesion at both shear rates tested. Incomplete inhibition under high shear is a plus in this case as the risk of bleeding upon treatment would be much lower than that of a direct inhibitor.

### Activators of Human VWF

Von Willebrand disease (VWD) is reportedly the most common inherited bleeding disorder and can also arise as an acquired syndrome (i.e. acquired von Willebrand syndrome, AVWS). These disorders develop due to defects and/or deficiency of the plasma protein von Willebrand factor (VWF). Over-expression or increased activity of VWF is also a major factor in many thrombotic disorders.

The VWF activity assay is used in clinical diagnostics of many bleeding and thrombotic disorders. Ristocetin, a small-molecule glycopeptide, is used clinically to determine VWF activity assay, or commonly known as the ristocetin cofactor (VWF: RCo) assay. However, ristocetin tends to flocculate other proteins and certain VWF: RCo assays use a turbidometric or aggregometer readout and has been difficult to standardize. Like ristocetin, certain polypeptides disclosed herein activate VWF by a similar mechanism; thus, may be used in VWF activity assays as a substitute for ristocetin.

The AIM can be disrupted by a number of factors, including a ristocetin-mimicking antibody 6G1. See DeLuca et al. Blood, 2000, 95 (1): 164-72. 6G1 binds to residues 1463-1472 in human VWF. Experiments indicate that ristocetin also binds to the same region in the C-terminal part of the AIM (CAIM). Binding of 6G1 disrupts the integrity of the AIM and activates A1 binding to platelets. However, 6G1 binding to its epitope sequence is not strong enough to enable its binding to and activation of full-length VWF.

A recombinant protein containing human VWF residues 1238-1481 was generated from transfected human embryonic kidney cells. The protein was then injected into a llama. Peripheral blood mononuclear cells were isolated. Immunoglobulin single domain cDNA genes were subcloned into pYDNB, a derivative of the pCTcon2 plasmid, and for expression in a yeast display library. The library was screened against recombinant proteins containing human VWF residues 1238-1461 (CAIM-less) or 1268-1493 (NAIM-less), both of which contain activated A1 domain. The population that bound to 1238-1461 was depleted from the library, and the remaining population should only contain binders to residues 1462-1481. In parallel, the population that bound to 1268-1493 was depleted and the remaining population should contain binders to residues 1238-1267. The depleted library subsets were then screened for the following properties:
binding to recombinant protein containing human VWF residues 1238-1493 (i.e. full sequence of AIM-A1)
induction of high-affinity binding of the AIM-A1 protein to recombinant protein containing the C-terminal ligand-binding domain of human GPIbα.

Three clones, Cd1C4, Cd1C11, and Cd1D12, have been identified and sequenced. Since all of them are generated from the aforementioned negative screen against CAIM-less, epitopes are expected to be located in the N-terminal part of AIM (NAIM, residues 1238-1267). Each protein has been subcloned into the expression vector and produced from E. coli with a C-terminal His tag.

Both Cd1C11 and Cd1D12 can blot the AIM-A1 protein in a dot plot, indicating that they recognize the linear epitope in the protein.

Cd1C11 binds AIM-A1 with an affinity of 29 nM, and Cd1D12 binds AIM-A1 with an affinity of 76 nM, as detected by biolayer interferometry.

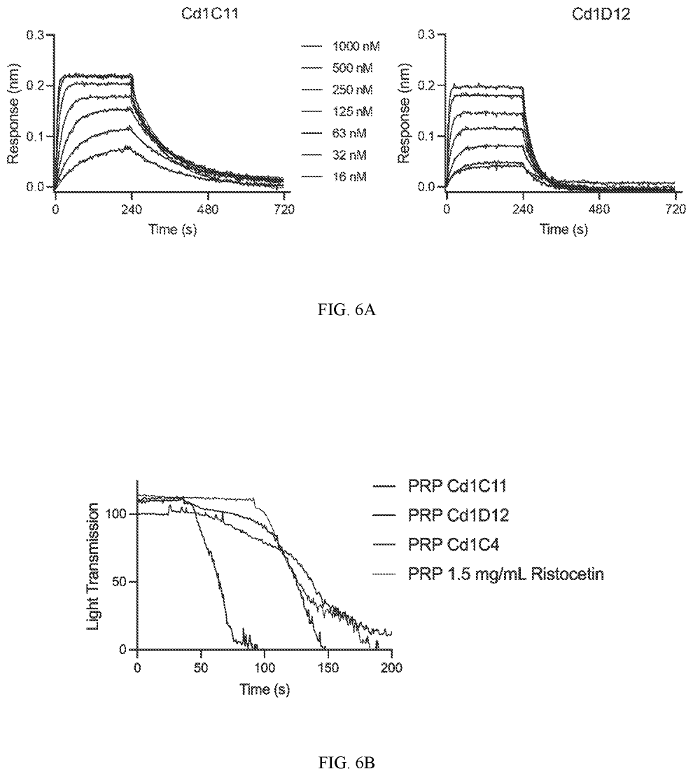

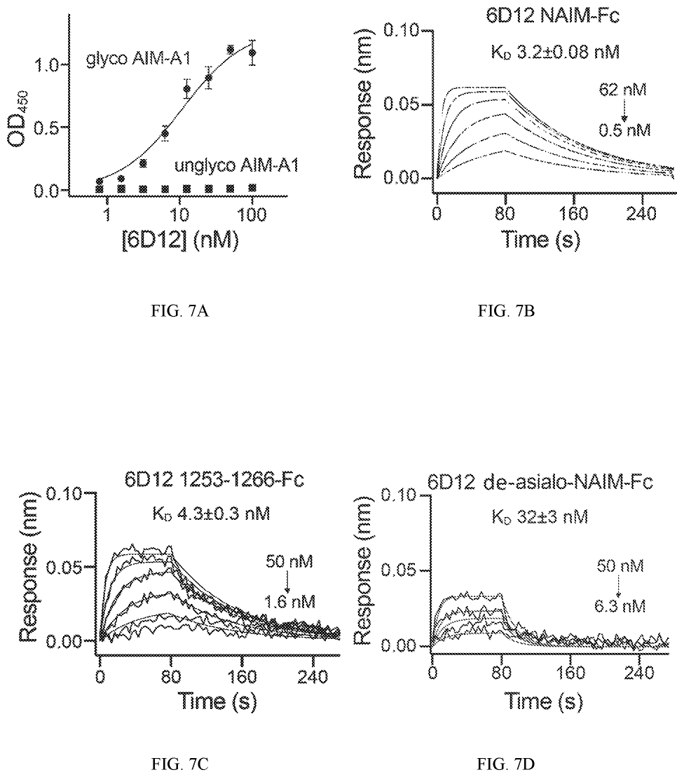

Addition of Cd1C4, Cd1C11 and Cd1D12 to human platelet-rich plasma induces rapid platelet aggregation, like ristocetin (FIGS. 6A and 6B). Protein sequences of VWF-activating ISVD antibodies are provided below.

Cd1C4 sequence:
(SEQ ID NO: 59)
MQVQLVESGG GLVQTGGSLT LSCAASGRAF SQYSVGWFRQ
APGKERTFVA AINWSGTKAY YAGSVKGRST ISRDRAKTTV
FLQMNSLKPE DTAVYYCATH RGLYYEGTNY SQKDEYDYWG
QGTQVTVSSE PKTPKPQPLE HHHHHH
Cd1C11 sequence:
(SEQ ID NO: 60)
MQVQLVESGG GLVQTGGSLR LSCAASGRTV SHYSVGWFRQ
APGKERVFVG AINWSGDKAY YVGSVKGRST IRRDNAKNTV
YLQMNSLKPE DTAVYYCATR RGLYYEGTDY SRKDEYDYWG
QGTQVTVSSE PKTPKPQPLE HHHHHH
Cd1D12 sequence:
(SEQ ID NO: 61)
MQVQLVESGG GLVQPGGSLR LSCAASGFTL DDYAIGWFRQ
VPGKERTFVG AINWSGTKAY YAGSVKGRST VRRDNAKNTV
YLQMNSLKPE DTAVYYCATH RGLHYGGINY SQKDEYDYWG
QGTQVTVSSE PKTPKPQPLE HHHHHH

### Nanobody Activators of Von Willebrand Factor Via Targeting Its Autoinhibitory Module

Cd1C4, Cd1C11, Cd1D12 are also called respectively 6C4, 6C11, and 6D12. R6Nd4 and R6Nd6 are also called Nd4 and Nd6, respectively. An AIM-A1-specific nanobody yeast display library was established. Several rounds of flow cytometry-based cell sorting of yeast cells with aforementioned binding properties produced AIM-binding nanobodies. Nanobodies encoded in three single clones have been expressed from E. coli and they exhibited differential binding affinities towards AIM-A1. Clone 6C4 showed an affinity of K_{D} 120 nM, 6D12 showed an affinity of K_{D} 31 nM, and 6C11 showed an affinity of K_{D} 13.5 nM. These nanobodies showed no detectable affinity towards recombinant A1-CAIM protein (residues 1268-1493), indicating that their epitopes are located in the N-terminal portion of the AIM (residues 1238-1267).

When added to human platelet-rich plasma, each nanobody dose-dependently activated platelets and rapidly induced full platelet aggregation at concentrations exceeding the affinity of the nanobody for VWF. The aggregation could be inhibited by the addition of antibodies that block the interaction between VWF and GPIbα. Plots of extents of aggregation as a function of nanobody concentration produced EC 50 values of about 100 nM for 6C11 and 6D12.

By isolating nanobodies that can bind specifically to the AIM and activate plasma VWF, experiments indicate that the AIM protects the A1 domain from binding to platelets. Interestingly, these nanobodies bind to the NAIM, on the opposite side of the module compared to ristocetin, the only known AIM-activating agent until now. With higher VWF-binding affinities than ristocetin and a robust profile as stable monomers, these nanobodies may prove useful in VWF-related research and diagnostics.

Nanobodies that Bind to Glycosylated NAIM Residues Distal to the A1 Domain

Experiments indicate that epitopes for 6C4, 6C11, and 6D12 are located in residues 1253-1266 of human VWF and contains part of sialylated O-glycans therein. To define the mechanism by which these nanobodies activate VWF, binding epitopes were characterized. Lack of binding of these nanobodies to A1-CAIM or tAIM-A1 suggests that epitopes include a sequence in the NAIM preceding residue 1261. The nanobodies could recognize denatured AIM-A1 protein in Western blot, regardless of its redox state, suggesting that they recognize a linear epitope in the NAIM.

The binding epitope of nanobody 6D12 was further defined. NAIM contains O-linked glycans. Thus, experiments were performed to determine whether the 6D12 epitope may include a glycan in the NAIM. Thus, unglycosylated AIM-A1 protein was produced from E. coli. There are multiple O-linked sialylated core 1 glycans in the AIM-A1 protein produced from transfected HEK293F cells. In comparison, 6D12 bound to glycosylated AIM-A1, with an apparent affinity around 10 nM. To test if the O-glycosylated NAIM contains the entire 6D12 epitope, IgG1-Fc fusion proteins that contained the NAIM sequence (residues 1238-1271), or a section therein that contained 3 O-glycosylation sites (residues 1253-1266), were expressed from transfected Expi293F cells. 6D12 exhibited tight binding to both NAIM-Fc and 1253-1266-Fc in BLI and Western blot, with binding affinities of 3.2 nM and 4.3 nM, respectively. Furthermore, 6D12 bound to desialylated NAIM-Fc, produced from neuraminidase treatment of NAIM-Fc, with an affinity of 32 nM. This is significantly weaker than that for NAIM-Fc. Overall, these results indicate that the 6D12 epitope is located in residues 1253-1266 (SPTTLYVEDISEPP, SEQ ID NO: 64) of the NAIM and contains part of sialylated O-glycans therein.

NAIM-Fc or 1253-1266-FC was immobilized on Protein A sensors binding to a serial dilution of 6C4, 6C11, or 6D12. Concentrations in serial dilutions were used to calculate affinity for each nanobody. Affinities and standard deviation of the measurement was determined through 1:1 global kinetic fitting to each trace with observable binding. Experiments using bio-layer interferometry sensorgrams also indicate that 6C4 and 6C11 also bind the O-glycosylated 1253-1266 sequence with high affinity, 14 nM and 4 nM respectively.

1-28. (canceled)

29. A polypeptide comprising an immunoglobulin single variable domain that specifically binds A1 or the autoinhibitory module on the C terminal end of the A1 domain in von Willebrand Factor (VWF) and having complementary determining region (CDR) CDR 1, CDR 2, and CDR 3 and framework regions (FRs), and wherein the CDR1, CDR 2, and CDR 3 are from camelid antibody R6Nd4.

30. The polypeptide of claim 29, having the following CDRs: a CDR1 of SRFSSRPMA (SEQ ID NO: 11), CDR2 of YINWSGGSKY (SEQ ID NO: 12); and a CDR3 of GRAYSAVAVTPRGYD (SEQ ID NO: 13).

31. The polypeptide of claim 30 having greater than 85% identity to
(SEQ ID NO: 3)
QVQLVESGGGLVQAGGSLRLSCAASGSRFSSRPMAWFRQTPGKEHDFVA
YINWSGGSKYYADSVKGRFTISRDNAKNTVYLQMDSLKPEDTSIYYCAA
GRAYSAVAVTPRGYDFWGQGTQVTVSS.

32. The polypeptide of claim 29 wherein the FRs have greater than 85% identity to the FRs in the parent sequence.

33. The polypeptide of claim 29 wherein the CDRs have greater than 85% identity to the CDRs in the parent sequence.

34. The polypeptide of claim 30, comprising two immunoglobulin single variable domains conjugated by a peptide linker or a disulfide bond from two cysteine amino acids.

35. A composition capable of reducing vWF activity comprising the polypeptide of claim 30.

36. A polypeptide comprising an immunoglobulin single variable domain that specifically binds A1 or the autoinhibitory module on the N terminal end of the A1 domain in von Willebrand Factor (VWF) having complementary determining region (CDR) CDR 1, CDR 2, and CDR 3 and framework regions (FRs), and wherein the CDR1, CDR 2, and CDR 3 are from at least one of camelid antibody Cd1C11 and Cd1D12.

37. The polypeptide of claim 36 wherein the camelid antibody is Cd1C11 having CDR1 with the amino acid sequence of RTVSHYSV (SEQ ID NO: 21), CDR2 of NWSGDKA (SEQ ID NO: 22); and CDR3 of RRGLYYEGTDYSRKD (SEQ ID NO: 23).

38. The polypeptide of claim 36 having greater than 85% identity to
(SEQ ID NO: 24)
QVQLVESGGGLVQTGGSLRLSCAASGRTVSHYSVGWFRQAPGKERVFVG
AINWSGDKAYYVGSVKGRSTIRRDNAKNTVYLQMNSLKPEDTAVYYCAT
RRGLYYEGTDYSRKDEYDYWGQGTQVTVSS.

39. The polypeptide of claim 36 wherein the camelid antibody is Cd1C12 having CDR1 with the amino acid sequence of FTLDDYAI (SEQ ID NO: 25), CDR2 of NWSGTKA (SEQ ID NO: 26); and CDR3 of RGLHYGGINYSQKD (SEQ ID NO: 27).

40. The polypeptide of claim 36 having greater than 85% identity to
(SEQ ID NO: 28)
QVQLVESGGGLVQPGGSLRLSCAASGFTLDDYAIGWFRQVPGKERTFVG
AINWSGTKAYYAGSVKGRSTVRRDNAKNTVYLQMNSLKPEDTAVYYCAT
HRGLHYGGINYSQKDEYDYWGQGTQVTVSS.

41. The polypeptide of claim 36 wherein the FRs have greater than 85% identity to the FRs in the parent sequence.

42. The polypeptide of claim 37 comprising two immunoglobulin single variable domains conjugated by a peptide linker or a disulfide bond from two cysteine amino acids.

43. A composition capable of activating vWF comprising the polypeptide of claim 37.

44. A synthetic peptide, the peptide consisting of amino acids SPTTLYVEDISEP (SEQ ID NO: 64).


SEQUENCE LISTING (64 sequences)

SEQ ID NO: 1 - protein, 127 aa, synthetic construct
QVQLVESGGGLVQAGGSLRLSCAASGLTFMDHVMGWFRQAPGKEREFVAAVGRSAIMRDYADLVKGRFTISRDNAKNTVYLQMDSLKFEDTAVYYCAARTPFPSDMTWSLPNDYIYWGQGAQVTVSS

SEQ ID NO: 2 - protein, 124 aa, synthetic construct
QVQLVESGGGLVQAGGSLRLSCAASGEKLTQYVVGWLRQAPGKEREFVASISRSGVFTNYADSVKGRFTTSKDNAKNTVYLQMNSLTPDDTAIYFCAADSRYSGTDWRVRGEYWGQGTQVTVSS

SEQ ID NO: 3 - protein, 125 aa, synthetic construct
QVQLVESGGGLVQAGGSLRLSCAASGSRFSSRPMAWFRQTPGKEHDFVAYINWSGGSKYYADSVKGRFTISRDNAKNTVYLQMDSLKPEDTSIYYCAAGRAYSAVAVTPRGYDFWGQGTQVTVSS

SEQ ID NO: 4 - protein, 117 aa, synthetic construct
QVQLVESGGGLVQAGGSLRLSCATSGIIFSVYHMGWFRQTPGKERELVALISLSGSSTDYADSVKGRFAISRDNAKDTVFLQMNTLKPEDTAVYYCAARLGSSWKYWGQGTQVTVSS

SEQ ID NO: 5 - protein, 8 aa, synthetic construct
LTFMDHVM

SEQ ID NO: 6 - protein, 10 aa, synthetic construct
AVGRSAIMRD

SEQ ID NO: 7 - protein, 17 aa, synthetic construct
RTPFPSDMTWSLPNDYI

SEQ ID NO: 8 - protein, 8 aa, synthetic construct
EKLTQYVV

SEQ ID NO: 9 - protein, 10 aa, synthetic construct
SISRSGVFTN

SEQ ID NO: 10 - protein, 14 aa, synthetic construct
DSRYSGTDWRVRGE

SEQ ID NO: 11 - protein, 9 aa, synthetic construct
SRFSSRPMA

SEQ ID NO: 12 - protein, 10 aa, synthetic construct
YINWSGGSKY

SEQ ID NO: 13 - protein, 15 aa, synthetic construct
GRAYSAVAVTPRGYD

SEQ ID NO: 14 - protein, 9 aa, synthetic construct
IIFSVYHMG

SEQ ID NO: 15 - protein, 10 aa, synthetic construct
LISLSGSSTD

SEQ ID NO: 16 - protein, 7 aa, synthetic construct
RLGSSWK

SEQ ID NO: 17 - protein, 8 aa, synthetic construct
RAFSQYSV

SEQ ID NO: 18 - protein, 7 aa, synthetic construct
NWSGTKA

SEQ ID NO: 19 - protein, 15 aa, synthetic construct
HRGLYYEGTNYSQKD

SEQ ID NO: 20 - protein, 128 aa, synthetic construct
QVQLVESGGGLVQTGGSLTLSCAASGRAFSQYSVGWFRQAPGKERTFVAAINWSGTKAYYAGSVKGRSTISRDRAKTTVFLQMNSLKPEDTAVYYCATHRGLYYEGTNYSQKDEYDYWGQGTQVTVSS

SEQ ID NO: 21 - protein, 8 aa, synthetic construct
RTVSHYSV

SEQ ID NO: 22 - protein, 7 aa, synthetic construct
NWSGDKA

SEQ ID NO: 23 - protein, 15 aa, synthetic construct
RRGLYYEGTDYSRKD

SEQ ID NO: 24 - protein, 128 aa, synthetic construct
QVQLVESGGGLVQTGGSLRLSCAASGRTVSHYSVGWFRQAPGKERVFVGAINWSGDKAYYVGSVKGRSTIRRDNAKNTVYLQMNSLKPEDTAVYYCATRRGLYYEGTDYSRKDEYDYWGQGTQVTVSS

SEQ ID NO: 25 - protein, 8 aa, synthetic construct
FTLDDYAI

SEQ ID NO: 26 - protein, 7 aa, synthetic construct
NWSGTKA

SEQ ID NO: 27 - protein, 14 aa, synthetic construct
RGLHYGGINYSQKD

SEQ ID NO: 28 - protein, 128 aa, synthetic construct
QVQLVESGGGLVQPGGSLRLSCAASGFTLDDYAIGWFRQVPGKERTFVGAINWSGTKAYYAGSVKGRSTVRRDNAKNTVYLQMNSLKPEDTAVYYCATHRGLHYGGINYSQKDEYDYWGQGTQVTVSS

SEQ ID NO: 29 - protein, 319 aa, synthetic construct
MPSSVSWGILLLAGLCCLVPVSLAERGSLNDIFEAQKIEWHTSQEPGGLWPPTDAPVSPTTLYVEDISEPPLHDFYCSRLLDLVFLLDGSSRLSEAEFEVLKAFVVDMMERLRISQKWVRVAWEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNHHHHHHHHHAHIVMVDAYKPTK

SEQ ID NO: 30 - protein, 34 aa, synthetic construct
QEPGGLVVPPTDAPVSPTTLYVEDISEPPLHDFY

SEQ ID NO: 31 - protein, 35 aa, synthetic construct
DLAPEAPPPTLPPDMAQVTVGPGLLGVSTLGPKRN

SEQ ID NO: 32 - protein, 128 aa, synthetic construct
EVQLVESGGGLVQPGGSLRLSCAASGRITSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 33 - protein, 5 aa, synthetic construct
GGGVV

SEQ ID NO: 34 - protein, 6 aa, synthetic construct
TALYYV

SEQ ID NO: 35 - protein, 5 aa, synthetic construct
TATYY

SEQ ID NO: 36 - protein, 5 aa, synthetic construct
VKVSS

SEQ ID NO: 37 - protein, 5 aa, synthetic construct
VTVKS

SEQ ID NO: 38 - protein, 5 aa, synthetic construct
VQVSS

SEQ ID NO: 39 - protein, 5 aa, synthetic construct
VTVQS

SEQ ID NO: 40 - protein, 5 aa, synthetic construct
VTVKS

SEQ ID NO: 41 - protein, 5 aa, synthetic construct
VTVQS

SEQ ID NO: 42 - protein, 5 aa, synthetic construct
VKVSS

SEQ ID NO: 43 - protein, 5 aa, synthetic construct
VQVSS

SEQ ID NO: 44 - protein, 6 aa, synthetic construct
VTVKSX

SEQ ID NO: 45 - protein, 6 aa, synthetic construct
VTVQSX

SEQ ID NO: 46 - protein, 6 aa, synthetic construct
VKVSSX

SEQ ID NO: 47 - protein, 6 aa, synthetic construct
VQVSSX

SEQ ID NO: 48 - protein, 6 aa, synthetic construct
VTVKSA

SEQ ID NO: 49 - protein, 6 aa, synthetic construct
VTVQSA

SEQ ID NO: 50 - protein, 6 aa, synthetic construct
VKVSSA

SEQ ID NO: 51 - protein, 6 aa, synthetic construct
VQVSSA

SEQ ID NO: 52 - protein, 6 aa, synthetic construct
VTVSSA

SEQ ID NO: 53 - protein, 259 aa, synthetic construct
EVQLVESGGGLVQPGGSLRLSCAASGRITSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 54 - protein, 13 aa, synthetic construct
AHIVMVDAYKPTK

SEQ ID NO: 55 - protein, 145 aa, synthetic construct
MQVQLVESGGGLVQAGGSLRLSCAASGLTFMDHVMGWFRQAPGKEREFVAAVGRSAIMRDYADLVKGRFTISRDNAKNTVYLQMDSLKFEDTAVYYCAARTPFPSDMTWSLPNDYIYWGQGAQVTVSSEPKTPKPQPLEHHHHHH

SEQ ID NO: 56 - protein, 142 aa, synthetic construct
MQVQLVESGGGLVQAGGSLRLSCAASGEKLTQYVVGWLRQAPGKEREFVASISRSGVFTNYADSVKGRFTTSKDNAKNTVYLQMNSLTPDDTAIYFCAADSRYSGTDWRVRGEYWGQGTQVTVSSEPKTPKPQPLEHHHHHH

SEQ ID NO: 57 - protein, 143 aa, synthetic construct
MQVQLVESGGGLVQAGGSLRLSCAASGSRFSSRPMAWFRQTPGKEHDFVAYINWSGGSKYYADSVKGRFTISRDNAKNTVYLQMDSLKPEDTSIYYCAAGRAYSAVAVTPRGYDFWGQGTQVTVSSEPKTPKPQPLEHHHHHH

SEQ ID NO: 58 - protein, 135 aa, synthetic construct
MQVQLVESGGGLVQAGGSLRLSCATSGIIFSVYHMGWFRQTPGKERELVALISLSGSSTDYADSVKGRFAISRDNAKDTVFLQMNTLKPEDTAVYYCAARLGSSWKYWGQGTQVTVSSEPKTPKPQPLEHHHHHH

SEQ ID NO: 59 - protein, 146 aa, synthetic construct
MQVQLVESGGGLVQTGGSLTLSCAASGRAFSQYSVGWFRQAPGKERTFVAAINWSGTKAYYAGSVKGRSTISRDRAKTTVFLQMNSLKPEDTAVYYCATHRGLYYEGTNYSQKDEYDYWGQGTQVTVSSEPKTPKPQPLEHHHHHH

SEQ ID NO: 60 - protein, 146 aa, synthetic construct
MQVQLVESGGGLVQTGGSLRLSCAASGRTVSHYSVGWFRQAPGKERVFVGAINWSGDKAYYVGSVKGRSTIRRDNAKNTVYLQMNSLKPEDTAVYYCATRRGLYYEGTDYSRKDEYDYWGQGTQVTVSSEPKTPKPQPLEHHHHHH

SEQ ID NO: 61 - protein, 146 aa, synthetic construct
MQVQLVESGGGLVQPGGSLRLSCAASGFTLDDYAIGWFRQVPGKERTFVGAINWSGTKAYYAGSVKGRSTVRRDNAKNTVYLQMNSLKPEDTAVYYCATHRGLHYGGINYSQKDEYDYWGQGTQVTVSSEPKTPKPQPLEHHHHHH

SEQ ID NO: 62 - protein, 88 aa, synthetic construct
VQLVESGGGLVQGGSLRLSCAASGSPMWFRQPGKVAIGGSYYDSVGRFTISRDNAKVYLQMSLEDTYYCAARAVPYFWGQGTQVTVSS

SEQ ID NO: 63 - protein, 87 aa, synthetic construct
VQLVESGGGLVQGGSLRLSCASGISMGWFRQPGKRELVAISGSTYDSVGRFISRDNAKVLQMNLEDTAVYYCAALSWGQGTQVTVSS

SEQ ID NO: 64 - protein, 13 aa, synthetic construct
SPTTLYVEDISEP